(19) United States
(12) Patent Application Publication
(10) Pub. No.: US 2026/0078402 A1
(43) Pub. Date: Mar. 19, 2026

(54) TRANSPOSON SYSTEM AND METHODS OF USING THE SAME

(71) Applicant: UPSIDE FOODS, INC., Berkeley, CA (US)

(72) Inventors: Ara Hwang, Berkeley, CA (US); Sukhdeep Singh Dhadwar, Berkeley, CA (US); Carmen Banks, Berkeley, CA (US); Rachel Michele Schumaker, Berkeley, CA (US)

(21) Appl. No.: 19/400,115

(22) Filed: Nov. 25, 2025

Related U.S. Application Data
(63) Continuation of application No. PCT/US2024/044647, filed on Aug. 30, 2024.
(60) Provisional application No. 63/580,265, filed on Sep. 1, 2023.

(51) Int. Cl. C12N 15/85; A23L 29/00; C12N 5/071; C12N 5/10

(52) U.S. Cl. CPC C12N 15/85; A23L 29/065; C12N 5/0602; C12N 5/10; C12N 2510/00; C12N 2800/90

(57) ABSTRACT

Provided herein are transposon/transposase systems for transposing and selecting of transposed cells where the system includes a transposase or polynucleotide encoding a transposase; and an exogenous nucleic acid sequence comprising: a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, where upon introducing (a) and (b) into a cell the transposon is transposed into the cell's genome and the polynucleotide encoding the selection marker is episomal.

### 1. CROSS REFERENCE TO RELATED APPLICATIONS

This application is a continuation of International Application No. PCT/US2024/044647, filed Aug. 30, 2024, which claims priority to U.S. Application No. 63/580,265, filed Sep. 1, 2023, each of which are hereby incorporated by reference in its entirety.

### 2. REFERENCE TO SEQUENCE LISTING

The contents of the electronic sequence listing having file name 38279_0015U2.xml; Size: 31,293 bytes; and Date of Creation: Nov. 24, 2025 is hereby incorporated by reference in its entirety.

### 3. FIELD OF THE INVENTION

Disclosed herein are edible cell based products comprising cells produced using vector systems appropriate for integrating nucleic acids encoding genes of interest into the genome of cells while encoded selectable markers remain episomal and are lost after selection of the integrated nucleic acids of interest and multiple cell population doublings. Cells lines and methods of producing cell based meat products from these cell lines are provided herein.

### 4. BACKGROUND

The mass production of cells remains limited by several factors, thus limiting final yields. Mass production of cells finds several downstream applications. For example, foods formulated from metazoan cells, cultured in vitro, have prospective advantages over their corporal-derived animal counterparts, including improved nutrition and safety. Production of these products have been projected to require fewer resources, convert biomass at a higher caloric efficiency and result in reduced environmental impacts relative to conventional in vivo methods. Together, metazoan cells, and their extracellular products, constitute a biomass that can potentially be harvested from a cultivation infrastructure for formulation of cell-based food products, such as cultured meat.

However, mass production of cells originating from cultured metazoan cells remains limited by several factors, for example, by senescence of the cells prior to reaching a density or number needed for a cell-based meat product, thus driving up the cost and resources needed to produce cell-based meat products. Provided herein are compositions and methods that address this and other related needs.

### 5. SUMMARY

This disclosure is based in part on the finding that including a polynucleotide sequence encoding a selection marker outside of the 5′ ITR and the 3′ ITR of a transposon and a transposase within the 5′ ITR and the 3′ ITR causes the transposase but not the selection marker to integrate into the cell. This phenomenon enables selection of the transposed cells via transiently expressed selection markers, while eliminating the need to further genetically modify a cell to remove a selection marker at a later time (see FIGS. 1-3). Applicant found that such a system enables cell line generation where selection markers are not required to be integrated into a cell's genome but rather exist in an episomal state, thereby allowing all the benefit of selection without the integration. This approach is particularly advantageous for cells destined to become food products, because the selection marker nucleic acid is lost relatively quickly and is therefore not intact when the cells are formulated into a food product. This approach thereby lessens the burden of creating a cell culture protocol that produces cells that are recognized as safe for consumption, because sequences encoding the selection marker are substantially and automatically eliminated during routine cell expansion and well before consumption. In one embodiment, the system includes a transposase or polynucleotide encoding a transposase; and an exogenous nucleic acid sequence comprising: a transposon comprising: a 5′ inverted terminal repeat (ITR), a first polynucleotide encoding a gene capable of modulating cell proliferation; and a 3′ inverted terminal repeat (ITR); and (ii) a polynucleotide encoding an at least first selection marker located outside of the 5′ ITR and 3′ ITR, where upon introducing (a) and (b) into a cell the transposon is transposed into the cell's genome and the polynucleotide encoding the selection marker is episomal. In cases where the first polynucleotide comprises a sequence coding for TERT or other genes with similar functions, this method increases a cell's replicative capacity, thereby increasing population doubling potential and reducing the number of starting cells needed in order to produce cell-based meat products.

Overall, this work demonstrated the ability to generate and select for cell lines having an integrated gene of interest without integrating selection markers that may not be recognized as safe for consumption. This approach is valuable because manufacturing cells suitable for consumption, i.e., those from commonly consumed animals, typically requires one or more cell line adaptations, such as adaptation into particular culture formats (e.g., suspension) and cell culture media, which require vast amounts of cells and extensive passaging and proliferation, e.g., population doubling levels (PDLs) of 100 or more, and this contributes to this technology field's tight constraints on economic feasibility.

In one aspect, this disclosure features a transposon system for transposing and selecting of transposed cells, the system comprising: (a) a transposase; and (b) an exogenous nucleic acid sequence comprising a first polynucleotide encoding a gene of interest located between a 5′ inverted terminal repeat (ITR) and a 3′ inverted terminal repeat (ITR), and a second polynucleotide encoding a selection marker located outside the 5′ ITR and the 3′ ITR; wherein the transposase integrates the first polynucleotide while leaving the second polynucleotide episomal.

In another aspect, this disclosure features a vector for transposing and selecting of transposed cells comprising: (a) a first polynucleotide sequence encoding a transposon comprising: a 5′ inverted terminal repeat (ITR), a first polynucleotide encoding a gene of interest; and a 3′ inverted terminal repeat (ITR); and (b) a second polynucleotide encoding a selection marker located outside of the 5′ ITR and 3′ ITR; and wherein upon introducing the vector into a cell comprising a suitable transposase the transposon is transposed into the cell's genome leaving the second polynucleotide episomal.

In some embodiments, the first polynucleotide encoding the gene of interest, the second polynucleotide encoding the selection marker, or both are operably linked to a first promoter.

In some embodiments, the first polynucleotide and second nucleotide are covalently linked on a circular nucleic acid vector, such as a plasmid.

In some embodiments, the transposon comprises a third polynucleotide encoding a second gene of interest, wherein the third polynucleotide encoding a gene of interest is located between the 5′ ITR and the 3′ ITR.

In some embodiments, the third polynucleotide encoding the second gene of interest is operably coupled to the first gene of interest or a second expression control element.

In some embodiments, the second gene of interest is selected from: an antibiotic resistance gene, a fluorophore, or a cell surface marker.

In some embodiments, where upon being introduced into the cell and following transposition, the episomal polynucleotide encoding the first selection marker remains in the cell for at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

In some embodiments, where, upon being introduced into the cell and following transposition, the episomal polynucleotide encoding the first selection marker remains in the cell for at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, or at least 30 days.

In another aspect, this disclosure features a method for generating a cell line suitable for dietary consumption, the method comprising: (a) introducing into a population of cells an exogenous nucleic acid sequence comprising a first polynucleotide encoding a gene of interest located between a 5′ inverted terminal repeat (ITR) and a 3′ inverted terminal repeat (ITR), and a second polynucleotide encoding a selection marker located outside of the 5′ ITR and the 3′ ITR, (b) selecting a transduced cell from the population of cells using the selection marker; and (c) culturing the transduced cells under conditions sufficient to expand the transduced cells. In embodiments, the first and second nucleic acids are within the same nucleic acid vector, including a transposon or transposon encoding vector, and including a circular nucleic acid vector.

In some embodiments, the method further comprises a second selecting step.

In some embodiments, the second selecting step comprises using the second selection marker to further select the transduced cell.

In some embodiments, the second selecting step is performed 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the first selecting step.

In some embodiments, the episomal polynucleotide encoding the selection marker remains in the cell following introduction for at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

In some embodiments, the cell line is derived from: livestock, poultry, or game animal species.

In some embodiments, the cell line comprises cells selected from: fibroblasts, mesenchymal cells, chondrocytes, myoblasts, myocytes, satellite cells, side population cells, muscle derived stem cells, myofibroblasts, mesenchymal stem cells, myogenic pericytes, or mesoangioblasts.

In another aspect, this disclosure features cell comprising: a genome having an integrated polynucleotide encoding a gene of interest between a 5′ ITR and a 3′ ITR, wherein the 5′ ITR and the 3′ ITR is transposable by a transpose; and an episomal polynucleotide encoding a selection marker.

In some embodiments, the gene of interest is selected from genes capable of modulating cell proliferation, a growth factor ligand, a growth factor receptor, a myogenic transcription factor, or a combination thereof.

In some embodiments, the selection marker is selected from: an essential nutrient, an antibiotic resistance gene, a fluorophore, or a cell surface marker.

### 6. BRIEF DESCRIPTION OF THE DRAWINGS

These and other features, aspects, and advantages of the present invention will become better understood with regard to the following description, and accompanying drawings, where:

FIG. 1 shows a schematic for transposition of the transposon comprising a promoter and a gene of interest into a cellular genome. ITR—inverted terminal repeats. btTERT—Bos taurus TERT. btCDK4^{R24C}. pA—polyA. AMP^{R}—ampicillin resistance. Ori—origin of replication. GFP-Puro—a green fluorescent protein (GFP) gene and a Puromycin resistance gene. Step 1 shows the transposition encoding the gene of interest between two ITRs and a selection marker outside of the two ITRs. Step 2 shows the integrated gene of interest.

FIG. 2 is a schematic showing a vector for use in transposition and selection of transposed cells. ITR—inverted terminal repeats. mRuby—polynucleotide encoding mRuby fluorescent protein. hEF1a—human EF1 alpha promoter. copGFP—a green fluorescent protein (GFP) gene. PuroR—a Puromycin resistance gene. SV40-polyA—a polyA sequence derived from SV40. 2A—self-cleavable peptide. BGH-poly—a polyA sequence derived from BGH. BtActin—Bos taurus actin promoter. Dotted line indicates a divide between the polynucleotide sequence encoding a transposon, which is integrated into a cellular genome, and the polynucleotide encoding a selection marker, which is not integrated into the cellular genome and thereby remains as an episome in the cell for at least some period of time, e.g. a transient expression period.

FIG. 3 illustrates one non-limiting example workflow of the methods described herein, including use of the vector described in FIG. 2 and expression and selection of transposed cells using one or both of the fluorescent protein encoded in the vector described in FIG. 2.

FIG. 4A shows a non-limiting experimental workflow used in Example 1 with chicken fibroblast cells transfected to integrate mRuby and transiently express copGFP.

FIG. 4B shows percent fluorescence of copGFP and mRuby when cells are transfected with a Donor Vector transposase integrating mRuby, while copGFP is only episomally delivered, at 3 days post transfection and again at 14 days post transfection.

FIG. 4C shows percent fluorescence of copGFP and mRuby when cells are transfected with a Donor Vector without a transposase for integrating mRuby, e.g. both genes are only delivered episomally. Data is shown for 3 days post transfection and again at 14 days post transfection.

FIG. 5A shows a non-limiting experimental workflow used in Example 2 with bovine fibroblast cells transfected to integrate mRuby and transiently express copGFP.

FIG. 5B shows percent fluorescent for copGFP and mRUBY for bovine fibroblast cells transfected with a Donor Vector transposase integrating mRuby, while copGFP is only episomally delivered. Data is shown for cells at 3 days, 10 days, 20 days, and 27 days post transfection.

FIG. 5C shows percent fluorescence of for copGFP and mRuby when cells are transfected with a Donor Vector without a transposase. Data is shown for 3, 15, and 20 days post transfection, whereupon episomally delivered gene expression reaches near zero.

FIGS. 6A-6C show an example workflow used to isolate GFP⁻ cells. FIG. 6A shows the experimental outline. FIG. 6B highlights the GFP⁻ cells to be isolated. FIG. 6C quantifies isolated cells at 14 days and at 21 days for fluorescence, showing very little episomal GFP at 21 days.

FIG. 7 is a schematic of a donor vector that includes TERT and CDK4 genes inside the ITR sequences of the transposon and copGFP and PuroR selection markers outside the ITR sequences.

FIG. 8 provides a PDL count and doubling time (DT) average across three cell lines, each having different parental cells and each with genes of interest integrated via transposase and selected via transiently expressed selection markers.

FIGS. 9A-9C show the results for the bovine cell lines transfected with transposase genes to generate immortalized cell lines, e.g. PDL 100+, as compared to controls. FIGS. 9A-9C show copy number quantification of TERT, CDK4 or PuroR (plasmid backbone component) genes for cell lines 10f, 13i, and 15E several passages after they have been transfected by a vector that integrates TERT/CDK4 and episomally delivers PuromycinR for transient expression.

FIGS. 10A-10C show copy number quantification of TERT, CDK4 or PuroR (plasmid backbone component) genes for cell lines 10F, 13i, and 15E 12 to 15 PDLs after they have been transfected by a transposon system, which integrates T/C and transiently expresses PuroR. FIG. 10A shows copy numbers of the 10F T/C cell line. In this example, 0.3 copies of puromycin resistant backbone fraction remains, and this quantity is expected to reach zero with further passaging, while TERT is integrated. FIG. 10B shows copy numbers of 13i T/C cell line. FIG. 10C shows copy numbers of 15E T/C cell line.

FIGS. 11A-11D show procurement and growth of cells into a cell mass suitable for consumption.

FIG. 12 shows copy number quantification of TERT, CDK4, and PuroR in a 13E cell line at 80 PDL and 160 PDL.

### 7. DETAILED DESCRIPTION

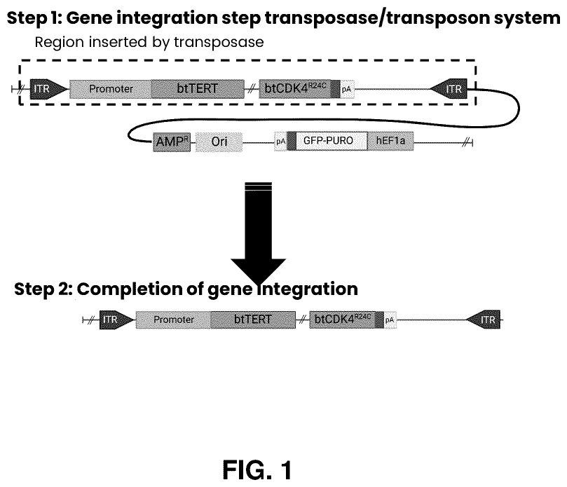

Provided herein are transposon/transposase systems for transposing and selecting of transposed cells. In particular, this disclosure is based in part on the finding that including a polynucleotide sequence encoding a selection marker outside of the 5′ ITR and the 3′ ITR, the selection marker does not integrate into the cell upon being introduced with the transposes and that this phenomenon enables selection of the transposed cells (see FIGS. 1-3). This enables cell line generation where selection markers are not required to be integrated into a cell's genome but rather exist in an episomal state, thereby allowing all the benefits of targeted, efficient selection without the integration. In one non-limiting example, a system includes a transposase or polynucleotide encoding a transposase; and an exogenous nucleic acid sequence comprising: a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, where upon introducing (a) and (b) into a cell the transposon is transposed into the cell's genome and the polynucleotide encoding the selection marker is episomal. Such a system provides a transiently selectable cell(s), wherein the cell(s) is selectable for a time period that coincides with a duration (i.e. lifespan) for which the episomal portion retains its structural and functional integrity. In some cases, the gene of interest is a gene capable of modulating cell proliferation. The resulting cell line is then capable of expanding at a scale required for cell-based meat products suitable for consumption.

### 7.1. Definitions

Unless defined otherwise, all technical and scientific terms used herein have the same meaning as is commonly understood by one of ordinary skill in the art to which this disclosure belongs. In the event that there is a plurality of definitions for terms cited herein, those in this section prevail unless otherwise stated.

Throughout this disclosure, the term “a” or “an” entity refers to one or more of that entity. As such, the terms “a” (or “an”), “one or more,” and “at least one” can be used interchangeably herein. Furthermore, “and/or” as used in a phase such as “A and/or B” herein is intended to include “A and B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “comprising” and “including” or grammatical variants thereof are to be taken as specifying the stated features, integers, steps, or components but do not preclude the addition of one or more additional features, integers, steps, components, or groups thereof. This term encompasses the terms “consisting of” and “consisting essentially of”.

As used herein, the terms “cell” and “cell line” are sometimes used interchangeably. As used herein, the term “cell” can refer to one or more cells originating from a cell line. As used herein, the term “cell line” can refer to a population of cells.

As used herein, the term “myoblast” refers to mononucleated muscle cells. They are embryonic precursors of myocytes, also called muscle cells. Although myoblasts may be classified as skeletal muscle myoblasts, smooth muscle myoblasts, and cardiac muscle myoblasts depending on the type of muscle cell that they will differentiate into, in this specification the term myoblasts refer to skeletal muscle myoblasts.

As used herein, the term “myotube” refers to elongated structures, the result of differentiated myoblast. Upon differentiation, myoblasts fuse into one or more nucleated myotubes and express skeletal muscle markers.

As used herein, the terms “immortalized cell” or “immortalized cell line” refer to cells that are passaged or modified to proliferate indefinitely and evade normal cellular senescence.

As used herein, the term “population doubling level (PDL)” refers to the total number of times the cells in the population have doubled since their primary isolation in vitro. Mathematically this is described as n=3.32 (log UCY−log 1)+X, where n=the final PDL number at end of a given subculture, UCY=the cell yield at that point, 1=the cell number used as inoculum to begin that subculture, and X=the doubling level of the inoculum used to initiate the subculture being quantitated.

As used herein the term “passaged cell” refers to the number of times the cells in the culture have been subcultured. This may occur without consideration of the inoculation densities or recoveries involved.

As used herein, the term “fragment” or “portion” when referring to a protein or a polynucleotide refers to a protein that comprises a domain, portion, or fragment of a parent or reference protein or polypeptide. The term “portion” can be used interchangeably with the term “functional portion.” The term “fragment” can be used interchangeably with the term “functional fragment.” The terms “functional portion” or “functional fragment” retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent protein or polypeptide, or provides a biological benefit. A “functional portion” or “functional fragment” of a protein or polypeptide has “similar binding” or “similar activity” when the functional portion or fragment displays no more than a 50% reduction in performance in a selected assay as compared to the parent or reference protein or polypeptide (preferably no more than 20% or 10%, or no more than a log difference as compared to the parent or reference with regard to affinity).

As used herein, the term “substantially free of” or “substantially free from” means the amount (e.g., absolute number within a population or concentration/percentage within a population) of a cell or cell type is below a value where the cell or cell type, or any cell derived therefrom, could contribute to the population. For example, a population substantially free of a cell means that upon differentiation of the population the cell does not contribute progeny to the differentiated population.

As used herein, the terms “transformed,” “transduced,” and “transfected” are used interchangeably unless otherwise noted. Each term refers to introduction of a nucleic acid sequence or polypeptide into a cell (e.g., an immortalized cell).

As used herein, the term “transposase” refers to a polypeptide that catalyzes the excision of a corresponding transposon from a donor polynucleotide, for example a vector, and the subsequent integration of the transposon into a sequence in a cell's genome.

As used herein, the term “transposition” refers to the action of a transposase in excising a transposon from one polynucleotide and then integrating it, either into a different site in the same polynucleotide, or into a second polynucleotide (e.g., a cell's genome).

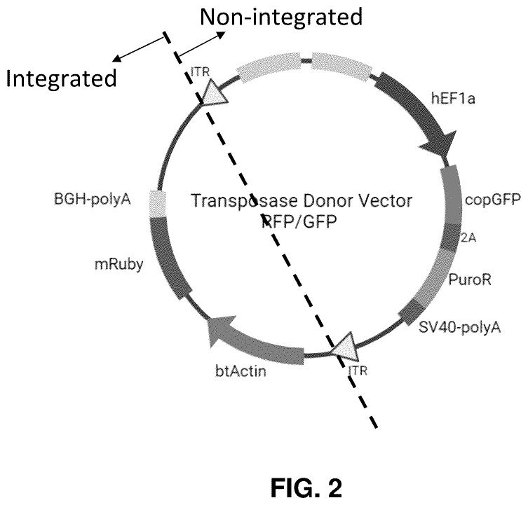

As used herein, the terms “outside the 5′ ITR and 3′ ITR” means with respect to a nucleic acid sequence in the context of the transposon or transposon encoding vector that the nucleic acid sequence is present at a position that is 5′ of the 5′ ITR sequence and at a position that is 3′ of the 3′ ITR sequence (in the 5′ to 3′ orientation of the nucleic acid) such that the sequence which is “outside the 5′ ITR and 3′ ITR” in the transposon construct is not integrated into the genome upon transposition. See, e.g., FIG. 2 in which copGFP-2A-PuroR encoding sequence is “outside the 5′ ITR and 3′ ITR”.

As used herein, the term “inside the 5′ ITR and 3′ ITR” means with respect to a nucleic acid sequence in the context of the transposon or transposon encoding vector that the nucleic acid sequence is present at a position that is 3′ of the 5′ ITR sequence and at a position that is 5′ of the 3′ ITR such that the sequence which is inside the 5′ ITR and 3′ITR is integrated into the genome upon transposition. See. e.g., FIG. 2 in which the mRuby coding sequence is inside the 5′ ITR and the 3′ ITR.

As used herein, the term “transposon” means a polynucleotide that can be excised from a first polynucleotide, for instance, a vector, and be integrated into a second position in the same polynucleotide, or into a second polynucleotide, for instance, the genomic or extrachromosomal DNA of a cell, by the action of a corresponding transposase. A transposon comprises a first transposon end (e.g., a 5′ ITR) and a second transposon end (e.g., a 3′ ITR), which are polynucleotide sequences recognized by and transposed by a transposase. Exemplary ITR nucleic acid sequences include a 5′ITR having a nucleotide sequence of SEQ ID NO: 17 (and, in embodiments, its reverse complement) and a 3′ ITR having a nucleotide sequence of SEQ ID NO: 18 (and, in embodiments, its reverse complement). A transposon usually further comprises a first polynucleotide sequence between the two transposon ends (or “inside” the 5′ ITR and the 3′ITR), such that the first polynucleotide sequence is transposed along with the two transposon ends by the action of the transposase. This first polynucleotide in natural transposons frequently comprises an open reading frame encoding a corresponding transposase that recognizes and transposes the transposon. Transposons of the present invention are “synthetic transposons” comprising a heterologous polynucleotide sequence which is transposable by virtue of its juxtaposition between two transposon ends. Synthetic transposons may or may not further comprise flanking polynucleotide sequence(s) outside the transposon ends, such as a sequence encoding a transposase, a vector sequence or sequence encoding a selectable marker.

Technology descriptions may be presented as a series of steps to provide an example of one or more possible implementations. Unless evident from the context, the order in which these steps are presented does not necessarily imply a fixed chronological sequence of execution. Steps may be performed in a different order, concurrently, or iteratively and still fall within the scope of the present disclosure. In some instances, steps may be performed in parallel, while others may be performed sequentially. In other instances, specific steps may be repeated or omitted as needed, depending on context and purpose.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### 7.2. Transposon/Transposase Systems for Transposition and Selection of Transposed Cells

Provided herein is a transposon/transposase system for transposing and selecting of transposed cells, the system comprising: (a) a transposase or polynucleotide encoding a transposase; and (b) an exogenous nucleic acid sequence comprising: (i) a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, wherein the transposase is capable of recognizing the ITRs of the transposon, wherein upon introducing (a) and (b) into a cell the transposon is transposed into the cell's genome and the polynucleotide encoding the selection marker is episomal. In some embodiments, wherein, after transposing the first polynucleotide encoding the gene of interest into the cell and the gene of interest encodes a protein that is capable of modulating cell proliferation, the cell has an increased proliferation capacity as compared to a cell that does not have the first polynucleotide encoding the gene of interest integrated into the cell's genome. In some embodiments, wherein, after introducing the polynucleotide encoding the gene of interest (e.g., a gene capable of modulating cell proliferation), the population of cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more).

Provided herein is a transposon/transposase system for transposing and selecting of transposed cells, the system comprising: (a) a first vector comprising a polynucleotide encoding a transposase; and (b) a second vector comprising: (i) a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, wherein the transposase is capable of recognizing the ITRs of the transposon, wherein upon introducing (a) and (b) into a cell the transposon is transposed into the cell's genome and the polynucleotide encoding the selection marker is episomal. In some embodiments, wherein, after transposing the first polynucleotide encoding the gene of interest into the cell and the gene of interest encodes a protein that is capable of modulating cell proliferation, the cell has an increased proliferation capacity as compared to a cell that does not have the first polynucleotide encoding the gene of interest integrated into the cell's genome. In some embodiments, wherein, after introducing the polynucleotide encoding the gene of interest (e.g., a gene capable of modulating cell proliferation), the population of cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more).

Provided herein is a vector for transposing and selecting of transposed cells comprising: (a) a polynucleotide sequence encoding a transposon comprising: a 5′ inverted terminal repeat (ITR), a first polynucleotide encoding a gene of interest; and a 3′ inverted terminal repeat (ITR); and (b) a polynucleotide encoding an at least first selection marker located outside of the 5′ ITR and 3′ ITR; and (c) a polynucleotide encoding a transposase located outside of the 5′ ITR and 3′ ITR; wherein the transposase is capable of recognizing the ITRs of the transposon, wherein upon introducing the vector into a cell the transposon is transposed into the cell's genome and the polynucleotide encoding the selection marker is episomal. In some embodiments, wherein, after transposing the first polynucleotide encoding the gene of interest into the cell and the gene of interest encodes a protein that is capable of modulating cell proliferation, the cell has an increased proliferation capacity as compared to a cell that does not have the first polynucleotide encoding the gene of interest integrated into the cell's genome. In some embodiments, wherein, after introducing the polynucleotide encoding the gene of interest (e.g., a gene capable of modulating cell proliferation), the population of cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more).

Provided herein is a set of vectors for transposing and selecting of transposed cells comprising: (a) a first vector comprising: (i) a polynucleotide sequence encoding a transposon, wherein the transposon comprises: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR; and (b) a second vector comprising a polynucleotide encoding a transposase, wherein the transposase is capable of recognizing the ITRs of the transposon, wherein upon introducing the set of vectors into a cell the transposon is transposed into the cell's genome and the polynucleotide encoding the selection marker is episomal. In some embodiments, wherein, after transposing the first polynucleotide encoding the gene of interest into the cell and the gene of interest encodes a protein that is capable of modulating cell proliferation, the cell has an increased proliferation capacity as compared to a cell that does not have the first polynucleotide encoding the gene of interest integrated into the cell's genome. In some embodiments, wherein, after introducing the polynucleotide encoding the gene of interest (e.g., a gene capable of modulating cell proliferation), the population of cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more).

In some embodiments, wherein, upon being introduced into the cell and following transposition, the episomal polynucleotide encoding the first selection marker remains in the cell for at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

In some embodiments, wherein, upon being introduced into the cell and following transposition, the episomal polynucleotide encoding the first selection marker remains in the cell for at least 5 days, at least 6 days, at least 7 days, at least 8 days, at least 9 days, at least 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

### 7.3. Method for Generating a Cell Line Using a Transposon System

Provided herein is a method for generating a cell line (e.g., a cell line suitable for dietary consumption), the method comprising: (a) introducing into a population of cells a transposon/transposase system comprising a transposase or polynucleotide encoding a transposase; and an exogenous nucleic acid sequence comprising: (i) a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, wherein the transposase is capable of recognizing the ITRs of the transposon, wherein upon introducing the transposase (or polynucleotide encoding the transposase and the exogenous nucleic acid into a cell) the transposon is transposed into the cell's genome and the polynucleotide encoding the selection marker is episomal; (b) selecting step (a) transduced cells from the population of cells using the first selection marker; and (c) culturing the transduced cells under condition sufficient to expand the transduced cells. In some embodiments of the method described herein, wherein, after transposing the first polynucleotide encoding the gene of interest into the cell and the gene of interest encodes a protein that is capable of modulating cell proliferation, the cell has an increased proliferation capacity as compared to a cell that does not have the first polynucleotide encoding the gene of interest integrated into the cell's genome. In one embodiment, wherein after introducing the polynucleotide encoding the gene of interest (e.g., a gene capable of modulating cell proliferation), the population of cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more).

Provided herein is a method for generating a cell line (e.g., a cell line suitable for dietary consumption), the method comprising: (a) introducing into a population of cells a first vector comprising a polynucleotide encoding a transposase; and a second vector comprising: (i) a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, wherein the transposase is capable of recognizing the ITRs of the transposon, wherein upon introducing the first vector and second vector into a cell the transposon is transposed into the cell's genome and the polynucleotide encoding the selection marker is episomal; (b) an at least first selecting step a transduced cell from the population of cells using the first selection marker; and (c) culturing the transduced cells under condition sufficient to expand the transduced cells. In some embodiments of the method described herein, wherein after transposing the first polynucleotide encoding the gene of interest into the cell and the gene of interest encodes a protein that is capable of modulating cell proliferation, the cell has an increased proliferation capacity as compared to a cell that does not have the first polynucleotide encoding the gene of interest integrated into the cell's genome. In one embodiment, wherein, after introducing the polynucleotide encoding the gene of interest (e.g., a gene capable of modulating cell proliferation), the population of cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more).

In some embodiments, the selecting step comprises culturing the cells under conditions sufficient for selection (e.g., in concentrations of antibiotics sufficient to remove cells not transduced with the system, vector, or sets of vectors or with cell culture media lacking an essential nutrient to starve cells not transduced with the system).

In some embodiments, the selecting step relies on the episomally-expressed selection marker for selecting the transposed cells.

In some embodiments, the method also includes a second selecting step. In such cases, the system, vector, or sets of vectors can include a second selection marker. In such embodiments, the second selecting step comprises using the second selection marker to further select the transduced cell. In some embodiments, the second selecting step comprises using the first selection marker.

In some embodiments, the second selecting step is performed 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the first selecting step.

In some embodiments, the episomal polynucleotide encoding the selection marker remains in the cell following introduction for at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

In some embodiments, prior to introducing (or incorporating) the gene of interest encoding a protein that is capable of modulating cell proliferation, the population of bovine cells have a population doubling level (PDL) of no more than 50.

In some embodiments, after introducing (or incorporating) the gene of interest encoding a protein that is capable of modulating cell proliferation, the population of bovine cells have a population doubling level (PDL) of at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99.

In some embodiments, the cell line is derived from: livestock, poultry, or game animal species. In some embodiments, the cell line is derived from: chicken, duck, or turkey. In some embodiments, the cell line is derived from livestock species. In some embodiments, the livestock species is bovine or porcine. In some embodiments, the bovine cells, chicken cells, or both are non-myogenic cells.

In some embodiments, the non-myogenic cells are selected from: fibroblasts, mesenchymal cells, and chondrocytes. In other embodiments, the cells are myogenic cells. In some embodiments, the myogenic cells are myoblasts, myocytes, satellite cells, side population cells, muscle derived stem cells, myofibroblasts, mesenchymal stem cells, myogenic pericytes, or mesoangioblasts.

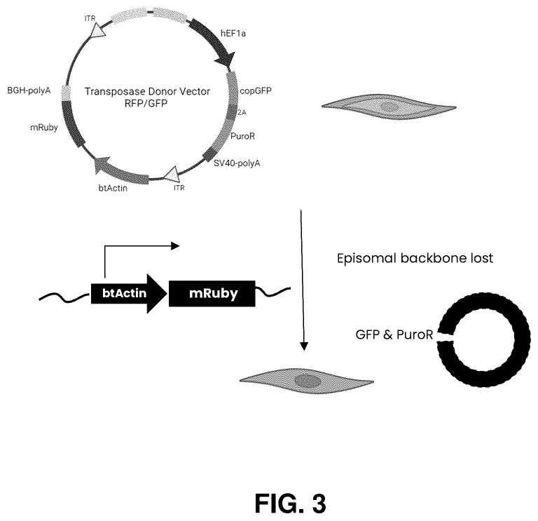

A non-limiting workflow of the methods described herein is as shown in FIG. 3.

### 7.4. Method of Increasing Cell Density

Provided herein are methods of increasing the cell density of a culture including the bovine cell line where the method includes introducing into, or incorporating into the genome of, a cell of the population of cells using a transposon system as described herein: a polynucleotide encoding telomere reverse transcriptase (TERT); and introducing into, or incorporating into the genome of, the cell containing the TERT polynucleotide at least one polynucleotide encoding a protein that is capable of modulating cell proliferation, including where the TERT polynucleotide and the polynucleotide encoding the protein that modulates cell proliferation are operably linked to regulatory elements that promote expression in the cells; and culturing the cells (e.g., bovine or chicken cells) in a cultivation infrastructure, thereby increasing the cell density of the culture as compared to controls.

In some embodiments, the method of increasing cell density includes use of the transposon system as described herein to incorporate a polynucleotide encoding TERT and at least one polynucleotide encoding a cell proliferation modulator to the cell genome such that TERT and the cell proliferation modulator are expressed from the integrated polynucleotides, e.g., are operably linked to heterologous promoters that promote expression in the cells.

In some embodiments, an increase in the cell density of a culture (e.g., suspension culture) using the methods described herein is about 1.025 fold, 1.05 fold, 1.10-fold, 1.15-fold, 1.20-fold, 1.25-fold, 1.30 fold, 1.35-fold, 1.40-fold, 1.45-fold, 1.5-fold, 2-fold, 2.5-fold, 3-fold, 3.5-fold, 4-fold, 4.5-fold, 5-fold, 7.5-fold, 10-fold, 15-fold, 20-fold, 25-fold, 30-fold, 40-fold, or even about 50-fold, 75-fold, 100-fold, 150-fold, or about 200-fold, compared to the density of a culture comprising bovine cells that do not include a non-native (i.e., having been introduced by the transposon system and/or under the control of a heterologous promoter) polynucleotide encoding telomere reverse transcriptase (TERT); and at least one non-native polynucleotide encoding a protein that is capable of modulating cell proliferation.

In some embodiments, an increase in the density of cells in a culture (e.g., suspension culture) using the methods described herein is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950^{%}, at least 1000%, compared to the density of a culture comprising bovine cells that do not include a non-native polynucleotide encoding telomere reverse transcriptase (TERT) and at least one non-native polynucleotide encoding a protein that is capable of modulating cell proliferation.

In some embodiments, methods described herein increase the density of cells in a culture of bovine cells by increasing the rate of proliferation of cells in the culture. In some embodiments, the increase in the rate of cell proliferation is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including values and ranges there between, compared to the density of a culture comprising bovine cells that do not include a non-native polynucleotide encoding telomere reverse transcriptase (TERT) and at least one non-native polynucleotide encoding a protein that is capable of modulating cell proliferation. In some embodiments, the increase in the rate of cell proliferation is about 25-1000%, about 25-750%, about 25-500%, about 50-1000%, about 50-750%, about 50-500%, about 100-1000%, about 100-750%, or about 100-500%, including values and ranges there between, compared to the density of a culture comprising bovine cells that do not include a non-native polynucleotide encoding telomere reverse transcriptase (TERT) and at least one non-native polynucleotide encoding a protein that is capable of modulating cell proliferation.

In some embodiments, methods described herein increase the cell density of a culture of bovine cells by decreasing cell death within the population of cells. In some embodiments, the decrease in cell death is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, including values and ranges there between, compared to the density of a comprising bovine cells that do not include a non-native polynucleotide encoding telomere reverse transcriptase (TERT) and at least one non-native polynucleotide encoding a protein that is capable of modulating cell proliferation. In some embodiments, a decrease in the rate of cell death within the population of bovine cells is about 2.5-10%, about 2.5-75%, about 2.5-50%, about 5.0-100%, about 5.0-75%, about 5.0-50%, about 10-100%, about 10-75%, or about 10-50%, including values and ranges there between, compared to the density of a culture comprising bovine cells that do not include a non-native polynucleotide encoding telomere reverse transcriptase (TERT) and at least one non-native polynucleotide encoding a protein that is capable of modulating cell proliferation.

In some embodiments, using the methods described herein, the density of cells in a culture may reach about 1E4 cells/mL, about 1E5 cells/mL, about 1E6 cells/mL, about 1E7 cells/mL, about 1E8 cells/mL, about 1E9 cells/mL, about 1E10 cells/mL, about 1E11 cells/mL, about 1E12 cells/mL, or about 1E13 cells/mL (cells in suspension culture or cells in the cellular biomass/mL of cultivation infrastructure), including values and ranges there between.

In some embodiments, using the methods described herein, the density of cells in a culture (e.g., suspension culture) may reach about 1 g/L, 5 g/L, 10 g/L, 25 g/L, 50 g/L, 75 g/L, 100 g/L, 150 g/L, 200 g/L, 250 g/L, 300 g/L, 350 g/L, 400 g/L, 450 g/L, 500 g/L, 550 g/L, 600 g/L, 650 g/L, 700 g/L, 750 g/L, 800 g/L, 850 g/L, 900 g/L, or 1000 g/L (g of cellular biomass/L of cultivation infrastructure), including values and ranges there between. In some embodiments, the density of cells in a culture (e.g., suspension culture) may range from about 1 g/L to about 5 g/L, about 1 g/L to about 750 g/L, about 1 g/L to about 500 g/L, about 1 g/L to about 250 g/L, about 1 g/L to about 100 g/L, about 1 g/L to about 50 g/L, about 5 g/L to about 1000 g/L, about 5 g/L to about 750 g/L, about 5 g/L to about 500 g/L, about 5 g/L to about 250 g/L, about 5 g/L to about 100 g/L, about 5 g/L to about 50 g/L, about 25 g/L to about 1000 g/L, about 25 g/L to about 750 g/L, about 25 g/L to about 500 g/L, about 25 g/L to about 300 g/L, about 25 g/L to about 250 g/L, about 25 g/L to about 100 g/L, about 50 g/L to about 1000 g/L, about 50 g/L to about 750 g/L, about 50 g/L to about 500 g/L, about 50 g/L to about 300 g/L, about 50 g/L to about 250 g/L, about 100 g/L to 1000 g/L, about 100 g/L to about 750 g/L, about 100 g/L to about 500 g/L, about 200 g/L to about 1000 g/L, about 200 g/L to about 750 g/L, about 200 g/L to about 500 g/L, about 300 g/L to about 1000 g/L, about 300 g/L to about 800 g/L, about 400 g/L to about 1000 g/L, or about 500 g/L to about 1000 g/L including values and ranges there between.

### 7.5. Methods for Producing Cell-Based Meat Suitable for Consumption

Provided herein are in vitro methods for producing cell-based meat suitable for consumption, comprising: (a) introducing into (or incorporating into the genome of) a cell of the population of cells a polynucleotide encoding telomere reverse transcriptase (TERT); (b) introducing into (or incorporating into the genome of) the cell of step (a) at least one polynucleotide encoding a protein that is capable of modulating cell proliferation and/or inducing myogenic specific differentiation to form myotubes, multinucleated myotubes, skeletal muscle fibers, and the like. In some embodiments, non-myogenic cells are used to produce a cell-based meat suitable for consumption with or without differentiation. In some embodiments, the in vitro method for producing cell-based meat suitable for consumption includes a step of adapting the cells to grow in suspension. In some embodiments, the in vitro method for producing cell-based meat suitable for consumption includes a step of culturing the cells in a cultivation infrastructure. In some embodiments, provided herein is cell-based meat suitable for consumption produced by the in vitro methods.

In some embodiments, the in vitro method for producing cell-based meat suitable for consumption includes using the transposon system to incorporate nucleic acids encoding TERT and at least one polynucleotide encoding a cell proliferation modulator to the cell genome of the cells which are cultured by methods described herein and used for the production of cell-based meat for consumption.

In some embodiments, the in vitro method for producing cell-based meat suitable for consumption includes introducing into the cell an agent (e.g., an effector protein) that induces a genetic perturbation in the integrated polynucleotide (e.g., the integrated polynucleotide encoding TERT or the integrated polynucleotide encoding the protein that is capable of modulating cell proliferation). In some embodiments, the agent is an effector selected from a nuclease, a recombinase, and an integrase.

In some embodiments, the in vitro method for producing cell-based meat suitable for consumption includes introducing into the cell a recombinase. In some embodiments, the recombinase is a CRE recombinase. In other embodiments, the present disclosure eliminates any need for CRE recombinase as commonly used.

Non-limiting examples of myogenic differentiation are as described in WO2019014652A1, WO2018208628A1, and WO2015066377A1, each of which is herein incorporated by reference in their entireties.

In some embodiments, the cultivated meat (also referred to as cultured meat, cell-based meat, lab grown meat, among others) produced according to the methods described herein can be processed as a raw, uncooked food product or as a cooked food product or as a cooked/uncooked food ingredient. In some embodiments, processing comprises withdrawal of the culture medium that supports the viability, survival, growth or expansion and differentiation, e.g., by starvation. Withdrawal may comprise physical removal of the culture medium or altering the composition of the culture medium, for example, by addition of components that would reduce or prevent further expansion and/or differentiation of the cell line or cells-derived from the cell line or by depletion of components that support expansion and/or differentiation of the cell line or cells derived from the cell line.

### 7.6. Transposon/Transposes Systems

In some embodiments, the transposase mediating transposition of the transposon is selected from commercially available options such that the corresponding 5′ ITR and 3′ ITR are matched to the transposase that is being used for the transposon.

In some embodiments, a transposase can be introduced into a cell as a protein or as a nucleic acid encoding the transposase, for example as a ribonucleic acid, including messenger RNA (mRNA) or any polynucleotide recognized by the transcriptional and/or translational machinery of a cell, as DNA, for example, as extrachromosomal DNA including episomal DNA, as plasmid DNA, or as viral nucleic acid. In some embodiments, the nucleic acid encoding the transposase can be transfected into a cell as a nucleic acid vector such as a plasmid, or as a gene expression vector, including a viral vector. The nucleic acid can be circular or linear. mRNA encoding the transposase may be prepared using DNA in which a gene encoding the transposase is operably linked to a heterologous promoter, such as the bacterial T7 promoter, which is active in vitro. DNA encoding the transposase protein can be stably inserted into the genome of the cell or into a vector for constitutive or inducible expression. In such cases where the transposase protein is transfected into the cell or inserted into the vector as DNA, the polynucleotide encoding the transposase is preferably operably linked to a promoter.

### 7.7. Genes Capable of Modulating Cell Proliferation

In some embodiments, the transposon includes a polynucleotide encoding a gene of interest, where the gene of interest encodes for a protein that is capable of modulating cell proliferation. In some embodiments, the protein that is capable of modulating cell proliferation is selected from TERT, BMI-1, CDK4, Cyclin D1, PCG1α, Nanog, DKC1 and YAP. Exemplary sequences provided in the Sequence Appendix.

### 7.7.1. Telomere Reverse Transcriptase (TERT)

In some embodiments, the polynucleotide encoding the gene of interest comprises a coding of telomerase reverse transcriptase (TERT). As used herein, “TERT” refers to telomerase reverse transcriptase (TERT) gene or TERT polypeptide that is a ribonucleoprotein polymerase that maintains telomere ends by addition of the telomere repeat TTAGGG. Telomerase expression plays a role in cellular senescence, as it is normally repressed in postnatal somatic cells resulting in progressive shortening of telomeres. In some embodiments, cells ectopically express the TERT polynucleotide. In some embodiments, the cells are genetically modified and carry stable integrations of one or more copies of the TERT polynucleotide. Exemplary methods for immortalizing a cell line are as described in WO2019014652A1, which is herein incorporated by reference in its entirety.

The polynucleotide encoding TERT can be from any organism. The TERT polynucleotide can be from bacteria, plants, fungi, and archaea. The TERT polynucleotide can be from any animal, such as vertebrate and invertebrate animal species. The TERT polynucleotide can be from any vertebrate animal species such as mammals, reptiles, birds, amphibians, and the like. The TERT polynucleotide can be from any mammalian species, such as a human, murine, bovine, porcine, and the like. In some embodiments, the coding sequence of the TERT protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix, Copra aegagrus hircus, or Homarus americanus. In some embodiments, the coding sequence of TERT protein is derived from Bos taurus.

In some embodiments, a polynucleotide comprising a coding sequence of TERT may encode any homolog of TERT, including TERT paralogs, or any other TERT paralogs, or an TERT protein translated from any splice variants of an TERT gene, or may comprise any mutations in the TERT gene sequence including, but not limited to nucleotide deletions, truncations, fusions, or substitutions. Mutations may be synthetic or naturally occurring.

In some embodiments, TERT refers to the TERT gene or TERT protein, or fragment or variant thereof (e.g., a TERT protein having one or more (e.g., one, two, three, four, five, six, seven, eight, nine, ten, or more amino acid substitutions, deletions or insertions as compared to a wild type TERT protein)).

In some embodiments, an TERT protein comprises an amino acid sequence having at least 80% (e.g., at least 85%, 90, 95%, 96%, 97%, 98%, or 99%) sequence identity to SEQ ID NO: 1. In some embodiments, the TERT protein sequence comprises an amino acid sequence of SEQ ID NO: 1.

In some embodiments, a polynucleotide encoding TERT comprises a nucleic acid sequence having at least 80% (e.g., at least 85%, 90, 95%, 96%, 97%, 98%, or 99%) sequence identity to SEQ ID NO: 2. In some embodiments, the polynucleotide encoding TERT comprises a nucleic acid sequence of SEQ ID NO: 2.

In such embodiments, one or more additional proteins that are capable of modulating cell proliferation can be introduced into the cell to achieve expansion of the replicative capacity of the cell. For example, a polynucleotide comprising the coding sequence of a BMI-1 protein, a CDK4 protein, a Cyclin D1 protein, a PCG1α protein, a DKC1 protein, a YAP1 protein, or a NANOG protein, or a combination thereof, can be introduced into the cell line to help immortalize or expand the replicative capacity of the cell.

### 7.7.1.1 Polycomb Complex Protein or Polycomb Ring Finger (BMI-1)

In some embodiments, the polynucleotide encoding the gene of interest comprises a coding sequence of polycomb complex protein or polycomb ring finger (BMI-1) or a fragment thereof. As used herein, “BMI-1” or “BMI1” refers to the polycomb complex protein or polycomb ring finger that is involved in mediating growth and development. Without wishing to be bound by theory, BMI-1 prevents senescence and immortalizes cells via telomerase activation. In some cases, elevation of Bmi-1 expression is closely correlated to the increased telomerase activity. BMI-1 aids in normal embryonic development and stem cell maintenance and is highly expressed in embryonic stem cells. Additionally, BMI-1 regulates the tumor suppressor proteins p16Ink4a and p14Arf, can promote CDK4 and CDK6 activity, directly regulates p53 stability, and maintains mitochondrial function and redox homeostasis. BMI-1 also is a key component in DNA Damage Response (DDR), for example, BMI-1 recruits the DDR machinery to DNA double-strand breaks (DSBs).

In some embodiments, the cells are modified to overexpress the coding sequence of a BMI-1 protein. In some embodiments, the cells are genetically modified and carry stable integrations of one or more copies of a BMI-1 coding sequence. In some embodiments, the cells overexpress the coding sequence of BMI-1 protein at levels sufficient to induce cell proliferation and/or immortalize the cell.

In some embodiments, the BMI-1 coding sequence is selected from any metazoan species. In some embodiments, the BMI-1 coding sequence is from any animal, such as vertebrate and invertebrate animal species. In some embodiments, the BMI-1 coding sequence is from any vertebrate animal species such as mammals, reptiles, birds, amphibians, and the like. In some embodiments, the BMI-1 coding sequence is from any mammalian species such as a human, murine, bovine, porcine, poultry, and the like. In some embodiments, the coding sequence of the BMI-1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of BMI-1 protein is derived from Bos taurus.

In some embodiments, a polynucleotide comprising a coding sequence of BMI-1 may encode any homolog of BMI-1, including BMI-1 paralogs, or any other BMI-1 paralogs, or a BMI-1 protein translated from any splice variants of a BMI-1 gene, or may comprise any mutations in the BMI-1 gene sequence including, but not limited to nucleotide deletions, truncations, fusions, or substitutions. Mutations may be synthetic or naturally occurring.

In some embodiments, BMI-1 refers to the BMI-1 gene or BMI-1 protein, or fragment or variant thereof (e.g., a BMI-1 protein having one or more (e.g., one, two, three, four, five, six, seven, eight, nine, ten, or more amino acid substitutions, deletions or insertions as compared to a wild type BMI-1 protein)).

In some embodiments, an BMI-1 protein comprises an amino acid sequence having at least 80% (e.g., at least 85%, 90, 95%, 96%, 97%, 98%, or 99%) sequence identity to SEQ ID NO: 3. In some embodiments, the BMI-1 protein sequence comprises an amino acid sequence of SEQ ID NO: 3.

In some embodiments, a polynucleotide encoding BMI-1 comprises a nucleic acid sequence having at least 80% (e.g., at least 85%, 90, 95%, 96%, 97%, 98%, or 99%) sequence identity to SEQ ID NO: 4. In some embodiments, the polynucleotide encoding BMI-1 comprises a nucleic acid sequence of SEQ ID NO: 4.

In some embodiments, one or more additional proteins that are capable of modulating cell proliferation can be introduced into the cell. For example, a polynucleotide comprising the coding sequence of a TERT protein, a CDK4 protein, a Cyclin D1 protein, a PCGlca protein, a DKC1 protein, a YAP1 protein, or a NANOG protein, or a combination thereof, can be introduced into the cell line to help immortalize or expand the replicative capacity of the cell.

### 7.7.2. Cyclin-Dependent Kinase 4 (CDK4)

In some embodiments, the polynucleotide encoding the gene of interest comprises a coding sequence of cyclin-dependent kinase 4 (CDK4) or a fragment thereof. Cyclin-dependent kinases are serine/threonine kinases whose activity depends on a regulatory subunit, for example, a cyclin (e.g., cyclin D1). The cyclin-dependent kinase family consists of CDK-1, CDK-4, and CDK-5, Cyclin-dependent kinases (CDKs). In some embodiments, the cyclin-dependent kinase is CDK4. In some embodiments, the cyclin-dependent kinase is a CKD1. In some embodiments, the cyclin-dependent kinase is a CDK5. As used herein, “CDK4” refers to the cyclin-dependent kinase (Cdk4) gene or CDK4 protein.

CDK4 phosphorylates cell cycle related proteins to promote cell cycle progression and regulates transcription of other genes to mediate cell proliferation. CDK4 is also involved in DNA damage repair and cancer cell survival.

In some embodiments, the cells are modified to overexpress the coding sequence of a cyclin-dependent kinase (e.g., CKD4, CDK1, and CDK5) protein. In some embodiments, the cells are genetically modified and carry stable integrations of one or more copies of a cyclin-dependent kinase coding sequence (e.g., one or more copies of a cyclin-dependent kinase coding sequence). In some embodiments, the cells overexpress the coding sequence of a cyclin-dependent kinase protein (e.g., CDK4, CDK1, and CDK5) at levels sufficient to increase cell proliferation and/or immortalize the cell.

In some embodiments, the cyclin-dependent kinase coding sequence is selected from any metazoan species. In some embodiments, the cyclin-dependent kinase coding sequence is from any animal, such as vertebrate and invertebrate animal species. In some embodiments, the cyclin-dependent kinase coding sequence is from any vertebrate animal species such as mammals, reptiles, birds, amphibians, and the like. In some embodiments, the cyclin-dependent kinase coding sequence is from any mammalian species such as a human, murine, bovine, porcine, poultry, and the like. In some embodiments, the coding sequence of the cyclin-dependent kinase protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the cyclin-dependent kinase protein is derived from Bos taurus.

In the methods described herein, a polynucleotide comprising a coding sequence of a cyclin-dependent kinase (e.g., a coding sequence of CDK4, a coding sequence of CDK1, and a coding sequence of CKD5). In some embodiments, a cyclin-dependent kinase may encode any homolog of a cyclin-dependent kinase, including cyclin-dependent kinase paralogs, or a cyclin-dependent kinase protein translated from any splice variants of a cyclin-dependent kinase gene, or may comprise any mutations in the cyclin-dependent kinase gene sequence including, but not limited to nucleotide deletions, truncations, fusions, or substitutions. Mutations may be synthetic or naturally occurring.

In some embodiments, a cyclin-dependent kinase protein (e.g., a CDK4 protein, a CDK1 protein, and a CDK6 protein) refers to the Cdk4 gene, Cdkl gene, and Cdk5 gene or CDK4 protein, CDK1 protein, or a CDK5 protein, respectively, or a fragment or variant thereof (e.g., a cyclin-dependent kinase protein having one or more (e.g., one, two, three, four, five, six, seven, eight, nine, ten, or more amino acid substitutions, deletions or insertions as compared to a wild type cyclin-dependent kinase protein)).

In some embodiments, a CDK4 protein comprises an amino acid sequence having at least 80% ((e.g., at least 85%, 90, 95%, 96%, 97%, 98%, or 99%) sequence identity to a sequence of SEQ ID NO: 5. In some embodiments, the CDK4 protein sequence comprises an amino acid sequence of SEQ ID NO: 5.

In some embodiments, a polynucleotide encoding CDK4 comprises a nucleic acid sequence having at least 80% (e.g., at least 85%, 90, 95%, 96%, 97%, 98%, or 99%) sequence identity to a sequence of SEQ ID NO: 6. In some embodiments, the polynucleotide encoding CDK4 comprises a nucleic acid sequence of SEQ ID NO: 6.

In some embodiments, introducing the polynucleotide comprising the coding sequence of the cyclin-dependent kinase protein (e.g., CDK4 protein, CDK1 protein, and a CDK5 protein) alone is not sufficient to increase cell proliferation and/or immortalize the cell. In such embodiments, one or more additional proteins that are capable of modulating cell proliferation can be introduced into the cell. For example, a polynucleotide comprising the coding sequence of a TERT protein, a BMI-1 protein, a Cyclin D1 protein, a PCG1α protein, a DKC1 protein, a YAP1 protein, or a NANOG protein, or a combination thereof, can be introduced into the cell line to help immortalize or expand the replicative capacity of the cell.

### 7.7.3. Cyclin D1

In some embodiments, the polynucleotide encoding the gene of interest comprises a coding sequence of cyclin D1 (CCND1) or a fragment thereof. As used herein, “cyclin D1” or “CYCLIND1” or “CCND1” refers to the cyclin D1 (Ccndl) gene or CCND1/cyclin D1 protein. Without wishing to be bound by theory, cyclin D1 proto-oncogene is an important regulator of G1 to S phase progression in many different cell types. Cyclin D1, together with its binding partners cyclin dependent kinase 4 and 6 (CDK4 and CDK6), it forms active complexes that promote cell cycle progression by phosphorylating and inactivating the retinoblastoma protein (RB). Without wishing to be bound by theory, cyclin D1 can also function as transcriptional modulator by regulating the activity of several transcription factors and histone deacetylase (HDAC3), which can be independent of CDK4 activity.

In some embodiments, the cells are modified to overexpress the coding sequence of a cyclin D1 protein. In some embodiments, the cells are genetically modified and carry stable integrations of one or more copies of a cyclin D1 coding sequence. In some embodiments, the cells overexpress the coding sequence of a cyclin D1 protein at levels sufficient to increase proliferation and/or immortalize the cell.

In some embodiments, the cyclin D1 coding sequence is selected from any metazoan species. In some embodiments, the cyclin D1 coding sequence is from any animal, such as vertebrate and invertebrate animal species. In some embodiments, the cyclin D1 coding sequence is from any vertebrate animal species such as mammals, reptiles, birds, amphibians, and the like. In some embodiments, the cyclin D1 coding sequence is from any mammalian species such as a human, murine, bovine, porcine, poultry, and the like. In some embodiments, the coding sequence of the cyclin D1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the cyclin D1 protein is derived from Bos Taurus.

In the methods described herein, a polynucleotide comprising a coding sequence of cyclin D1 may encode any homolog of cyclin D1, including cyclin D1 paralogs, or a cyclin D1 protein translated from any splice variants of a cyclin D1 gene, or may comprise any mutations in the cyclin D1 gene sequence including, but not limited to nucleotide deletions, truncations, fusions, or substitutions. Mutations may be synthetic or naturally occurring.

In some embodiments, cyclin D1 refers to the cyclin D1 gene or cyclin D1 protein, or fragment or variant thereof (e.g., a cyclin D1 protein having one or more (e.g., one, two, three, four, five, six, seven, eight, nine, ten, or more amino acid substitutions, deletions or insertions as compared to a wild type cyclin D1 polypeptide)).

In some embodiments, a cyclin D1 protein comprises an amino acid sequence having at least 80% ((e.g., at least 85%, 90, 95%, 96%, 97%, 98%, or 99%) sequence identity to a sequence of SEQ ID NO: 7. In some embodiments, the cyclin D1 protein sequence comprises an amino acid sequence of SEQ ID NO: 7.

In some embodiments, a polynucleotide encoding cyclin D1 comprises a nucleic acid sequence having at least 80% (e.g., at least 85%, 90, 95%, 96%, 97%, 98%, or 99%) sequence identity to SEQ ID NO: 8. In some embodiments, the polynucleotide encoding cyclin D1 comprises a nucleic acid sequence of SEQ ID NO: 8.

In some embodiments, one or more additional proteins that are capable of modulating cell proliferation can be introduced into the cell line. For example, a polynucleotide comprising the coding sequence of a TERT protein, a BMI-1 protein, a CDK4 protein, a PCG1α protein, a DKC1 protein, a YAP1 protein, or a Nanog, or a combination thereof, can be introduced into the cell line to help immortalize or expand the replicative capacity of the cell.

### 7.7.4. Peroxisome Proliferator-Activated Receptor Gamma Coactivator 1-Alpha (PGC-1α)

In some embodiments, the polynucleotide encoding the gene of interest comprises a coding sequence of peroxisome proliferator-activated receptor gamma coactivator 1-alpha (PGC-1α or PGC1-alpha) or a fragment thereof. As used herein, “PGC-1α” refers to the peroxisome proliferator-activated receptor gamma coactivator 1-alpha (PGC-1α) gene or PGC-1α.

Without wishing to be bound by theory, PGC1α interacts with PPAR7, which permits the interaction of this protein with multiple transcription factors. In some embodiments, PGC1α interacts with, and regulate the activities of, cAMP response element binding protein (CREB) and nuclear respiratory factors (NRFs). In some embodiments, PGC1α provides a direct link between external physiological stimuli and the regulation of mitochondrial biogenesis and is a major factor that regulates muscle fiber type determination.

In some embodiments, the cells are modified to overexpress the coding sequence of a PGC1α protein. In some embodiments, the cells are genetically modified and carry stable integrations of one or more copies of a PGC1α coding sequence. In some embodiments, the cells overexpress the coding sequence of PGC1α protein at levels sufficient to increase cell proliferation and/or immortalize the cell.

In some embodiments, the PGC1α coding sequence is selected from any metazoan species. In some embodiments, the PGC1α coding sequence is from any animal, such as vertebrate and invertebrate animal species. In some embodiments, the PGC1α coding sequence is from any vertebrate animal species such as mammals, reptiles, birds, amphibians, and the like. In some embodiments, the PGC1α coding sequence is from any mammalian species such as a human, murine, bovine, porcine, poultry, and the like. In some embodiments, the coding sequence of the PGC1α protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the PGC1α is derived from Bos taurus.

In the methods described herein, a polynucleotide comprising a coding sequence of PGC1α may encode any homolog of PGC1α, including PGC1α paralogs, or a PGC1α protein translated from any splice variants of a PGC1α gene, or may comprise any mutations in the PGC1a gene sequence including, but not limited to nucleotide deletions, truncations, fusions, or substitutions. Mutations may be synthetic or naturally occurring.

In some embodiments, PGC1α refers to the PGC1α gene or PGC1α protein, or fragment or variant thereof (e.g., a PGC1a protein having one or more (e.g., one, two, three, four, five, six, seven, eight, nine, ten, or more amino acid substitutions, deletions or insertions as compared to a wild type PGC1α polypeptide)).

In some embodiments, a PGC1α protein comprises an amino acid sequence having at least 80% ((e.g., at least 85%, 90, 95%, 96%, 97%, 98%, or 99%) sequence identity to a sequence of SEQ ID NO: 9. In some embodiments, the PGC1α protein sequence comprises an amino acid sequence of SEQ ID NO: 9.

In some embodiments, a polynucleotide encoding PGC1α comprises a nucleic acid sequence having at least 80% (e.g., at least 85%, 90, 95%, 96%, 97%, 98%, or 99%) sequence identity to sequence of SEQ ID NO: 10. In some embodiments, the polynucleotide encoding PGC1α comprises a nucleic acid sequence of SEQ ID NO: 10.

In some embodiments, introducing the polynucleotide comprising the coding sequence of the PGC1α protein alone is not sufficient to increase cell proliferation and/or immortalize the cell. In such embodiments, one or more additional proteins that are capable of modulating cell proliferation can be introduced into the cell. For example, a polynucleotide comprising the coding sequence of a TERT protein, a BMI-1 protein, a Cyclin D1 protein, a CDK4 protein, a DKC1 protein, a YAP1 protein, or a NANOG protein, or a combination thereof, can be introduced into the cell line to help immortalize or expand the replicative capacity of the cell.

### 7.7.5. Nanog

In some polynucleotide encoding the gene of interest comprises a coding sequence of Nanog (NANOG) or a fragment thereof. As used herein, “Nanog” refers to the Nanog gene or NANOG protein.

In some embodiments, the cells are modified to overexpress the coding sequence of a NANOG protein. In some embodiments, the cells are genetically modified and carry stable integrations of one or more copies of a Nanog coding sequence. In some embodiments, the cells overexpress the coding sequence of NANOG protein at levels sufficient to increase production and/or secretion of Nanog into the cell medium.

In some embodiments, the Nanog coding sequence is selected from any metazoan species. In some embodiments, the Nanog coding sequence is from any animal, such as vertebrate and invertebrate animal species. In some embodiments, the Nanog coding sequence is from any vertebrate animal species such as mammals, reptiles, birds, amphibians, and the like. In some embodiments, the Nanog coding sequence is from any mammalian species such as a human, murine, bovine, porcine, poultry, and the like. In some embodiments, the coding sequence of the NANOG protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the Nanog is derived from Bos taurus.

In the methods described herein, a polynucleotide comprising a coding sequence of Nanog may encode any homolog of Nanog, including Nanog paralogs, or a NANOG protein translated from any splice variants of a Nanog gene, or may comprise any mutations in the Nanog gene sequence including, but not limited to nucleotide deletions, truncations, fusions, or substitutions. Mutations may be synthetic or naturally occurring.

In some embodiments, NANOG refers to the Nanog gene or NANOG protein, or fragment or variant thereof (e.g., a NANOG protein having one or more (e.g., one, two, three, four, five, six, seven, eight, nine, ten, or more amino acid substitutions, deletions or insertions as compared to a wild type NANOG polypeptide)).

In some embodiments, a NANOG protein comprises an amino acid sequence having at least 80% ((e.g., at least 85%, 90, 95%, 96%, 97%, 98%, or 99%) sequence identity to a sequence of SEQ ID NO: 11. In some embodiments, the NANOG protein sequence comprises an amino acid sequence of SEQ ID NO: 11.

In some embodiments, a polynucleotide encoding NANOG comprises a nucleic acid sequence having at least 80% (e.g., at least 85%, 90, 95%, 96%, 97%, 98%, or 99%) sequence identity to SEQ ID NO: 12. In some embodiments, the polynucleotide encoding NANOG comprises a nucleic acid sequence of SEQ ID NO: 12.

In some embodiments, introducing the polynucleotide comprising the coding sequence of the NANOG protein alone is not sufficient to increase cell proliferation and/or immortalize the cell. In such embodiments, one or more additional proteins that are capable of modulating cell proliferation can be introduced into the cell line. For example, a polynucleotide comprising the coding sequence of a TERT protein, a BMI-1 protein, a CDK4, a Cyclin D1 protein, a PCG1α protein, a DKC1 protein, or a YAP protein, or a combination thereof, can be introduced into the cell line to help immortalize or expand the replicative capacity of the cell.

### 7.7.6. Dyskerin Pseudouridine Synthase 1 (DKC1)

In some embodiments, the polynucleotide encoding the gene of interest comprises a coding sequence of Dyskerin Pseudouridine Synthase 1 (DKC1) or a fragment thereof. As used herein, “DKC1” refers to the Dyskerin Pseudouridine Synthase 1 (DKC1) that plays an active role in telomerase stabilization and maintenance.

In some embodiments, the cells are modified to overexpress the coding sequence of a DKC1 protein. In some embodiments, the cells are genetically modified and carry stable integrations of one or more copies of a DKC1 coding sequence. In some embodiments, the cells overexpress the coding sequence of BMI-1 protein at levels sufficient to induce cell proliferation and/or immortalize the cell.

In some embodiments, the DKC1 coding sequence is selected from any metazoan species. In some embodiments, the DKC1 coding sequence is from any animal, such as vertebrate and invertebrate animal species. In some embodiments, the DKC1 coding sequence is from any vertebrate animal species such as mammals, reptiles, birds, amphibians, and the like. In some embodiments, the DKC1 coding sequence is from any mammalian species such as a human, murine, bovine, porcine, poultry, and the like. In some embodiments, the coding sequence of the DKC1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of DKC1 protein is derived from Bos taurus.

In some embodiments, a polynucleotide comprising a coding sequence of DKC1 may encode any homolog of DKC1, including DKC1 paralogs, or any other DKC1 paralogs, or an DKC1 protein translated from any splice variants of an DKC1 gene, or may comprise any mutations in the DKC1 gene sequence including, but not limited to nucleotide deletions, truncations, fusions, or substitutions. Mutations may be synthetic or naturally occurring.

In some embodiments, DKC1 refers to the DKC1 gene or DKC1 protein, or fragment or variant thereof (e.g., a DKC1 protein having one or more (e.g., one, two, three, four, five, six, seven, eight, nine, ten, or more amino acid substitutions, deletions or insertions as compared to a wild type DKC1 protein)). FIG. 15D shows a plasmid map of a construct used to introduce the coding sequence of BMI into any of the cells described herein.

In some embodiments, a DKC1 protein comprises an amino acid sequence having at least 80% (e.g., at least 85%, 90, 95%, 96%, 97%, 98%, or 99%) sequence identity to SEQ ID NO: 13. In some embodiments, the DKC1 protein sequence comprises an amino acid sequence of SEQ ID NO: 13.

In some embodiments, a polynucleotide encoding DKC1 comprises a nucleic acid sequence having at least 80% (e.g., at least 85%, 90, 95%, 96%, 97%, 98%, or 99%) sequence identity to SEQ ID NO: 14. In some embodiments, the polynucleotide encoding DKC1 comprises a nucleic acid sequence of SEQ ID NO: 14.

In some embodiments, introducing the polynucleotide comprising the coding sequence of the DKC1protein alone is not sufficient to immortalize the cell. In such embodiments, one or more additional proteins that are capable of modulating cell proliferation can be introduced into the cell. For example, a polynucleotide comprising the coding sequence of a TERT protein, a CDK4 protein, a Cyclin D1 protein, a PCG1α protein, a YAP, or a NANOG protein, or a combination thereof, can be introduced into the cell line to help immortalize or expand the replicative capacity of the cell.

### 7.7.7. Yes Associated Protein 1 (YAP1)

In some embodiments, the polynucleotide encoding the gene of interest comprises a coding sequence of Yes Associated Protein (YAP1) or a fragment thereof. As used herein, “YAP” or “YAP1” refers to the yes associated protein that is involved in mediating growth and development. In some embodiments, YAP1 expression (RNA and/or protein) levels are modulated as described in US20200165569A1, which is herein incorporated by reference in its entirety.

In some embodiments, the cells are modified to overexpress the coding sequence of a YAP1 protein. In some embodiments, the cells are genetically modified and carry stable integrations of one or more copies of a YAP1 coding sequence. In some embodiments, the cells overexpress the coding sequence of YAP1 protein at levels sufficient to induce cell proliferation and/or immortalize the cell.

In some embodiments, the YAP1 coding sequence is selected from any metazoan species. In some embodiments, the YAP1 coding sequence is from any animal, such as vertebrate and invertebrate animal species. In some embodiments, the YAP1 coding sequence is from any vertebrate animal species such as mammals, reptiles, birds, amphibians, and the like. In some embodiments, the YAP1 coding sequence is from any mammalian species such as a human, murine, bovine, porcine, poultry, and the like. In some embodiments, the coding sequence of the YAP1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of YAP1 protein is derived from Bos taurus.

In some embodiments, a polynucleotide comprising a coding sequence of YAP1 may encode any homolog of YAP1, including YAP1 paralogs, or any other BMI-1 paralogs, or an YAP1 protein translated from any splice variants of an YAP1 gene, or may comprise any mutations in the YAP1 gene sequence including, but not limited to nucleotide deletions, truncations, fusions, or substitutions. Mutations may be synthetic or naturally occurring.

In some embodiments, YAP1 refers to the YAP1 gene or YAP1 protein, or fragment or variant thereof (e.g., a YAP1 protein having one or more (e.g., one, two, three, four, five, six, seven, eight, nine, ten, or more amino acid substitutions, deletions or insertions as compared to a wild type YAP1 protein)).

In some embodiments, a YAP1 protein comprises an amino acid sequence having at least 80% (e.g., at least 85%, 90, 95%, 96%, 97%, 98%, or 99%) sequence identity to SEQ ID NO: 15. In some embodiments, the YAP1 protein sequence comprises an amino acid sequence of SEQ ID NO: 15.

In some embodiments, a polynucleotide encoding YAP1 comprises a nucleic acid sequence having at least 80% (e.g., at least 85%, 90, 95%, 96%, 97%, 98%, or 99%) sequence identity to a sequence of SEQ ID NO: 16. In some embodiments, the polynucleotide encoding YAP1 comprises a nucleic acid sequence of SEQ ID NO: 16.

In some embodiments, introducing the polynucleotide comprising the coding sequence of the YAP1 protein alone is not sufficient to immortalize the cell. In such embodiments, one or more additional proteins that are capable of modulating cell proliferation can be introduced into the cell. For example, a polynucleotide comprising the coding sequence of a TERT protein, a CDK4 protein, a Cyclin D1 protein, a PCGlca protein, a DKC1 protein, or a NANOG protein, or a combination thereof, can be introduced into the cell line to help immortalize or expand the replicative capacity of the cell.

### 7.8. Cells

In some embodiments, the bovine cells are non-myogenic cells. In some embodiments, the bovine cells are non-myogenic cells but harbor myogenic capacity. In some embodiments, the non-myogenic cells are fibroblasts, mesenchymal stem cells, and chondrocytes.

In some embodiments, the bovine cells are myogenic cells. In some embodiments, the myogenic cells are myoblasts, myocytes, satellite cells, side population cells, muscle derived stem cells, mesenchymal stem cells, myogenic pericytes, or mesoangioblasts. In some embodiments, the cells are non-myogenic cells.

### 7.9. Cultivation Infrastructure

In some embodiments, a cultivation infrastructure may be a tube, a cylinder, a flask, a petri-dish, a multi-well plate, a dish, a vat, a roller bottle, an incubator, a bioreactor, an industrial fermenter and the like.

In some embodiments, a cultivation infrastructure can be of any scale, and support any volume of cellular biomass and culturing reagents. In some embodiments, the cultivation infrastructure ranges from about 10 μL to about 100,000 L. In some embodiments, the cultivation infrastructure is about 10 μL, about 100 μL, about 1 mL, about 10 mL, about 100 mL, about 1 L, about 10 L, about 100 L, about 1000 L, about 10,000 L, or even about 100,000 L.

In some embodiments, the cultivation infrastructure comprises a substrate. In some embodiments, a cultivation infrastructure may comprise a permeable substrate (e.g. permeable to physiological solutions) or an impermeable substrate (e.g. impermeable to physiological solutions).

In some embodiments, the cultivation infrastructure comprises a primary substrate, which can be a flat, concave, or convex substrate. In some embodiments, the cultivation infrastructure further comprises a secondary substrate, either introduced, or autologous, to direct cellular growth between the substrates, e.g. to direct attachment, proliferation and hypertrophy of cells on a plane perpendicular to the primary substrate.

In some embodiments, the cultivation infrastructure comprises a hydrogel, a liquid cell culture media, or soft agar.

In some embodiments, the cultivation infrastructure does not comprise a substrate to which cells can adhere. In some embodiments, the cultivation infrastructure comprises a suspension culture, e.g. supporting the growth of a self-adhering biomass, or single-cell suspension in a liquid medium.

In some embodiments, the cultivation infrastructure comprises adherent cells (i.e. those cells that adhere to a substrate). In some embodiments, the cultivation infrastructure comprises non-adherent cells (i.e. those cells that do not adhere to a substrate).

Non-limiting examples of cultivation infrastructure include those described in U.S. Patent Publication Nos. 2020/0110347, 2022/0056394, and 2021/014031, which are herein incorporated by reference in their entireties.

### 7.10. Nucleic Acids/Vectors

In another aspect, provided herein are polynucleotides comprising coding sequences of any of the transposons described herein, including transposons having a 5′ inverted terminal repeat (ITR), a first polynucleotide encoding a gene of interest; and a 3′ inverted terminal repeat (ITR), any of the selection markers described herein (e.g., the first selection marker and the second selection marker), and any of the transposases described herein.

In another aspect, provided herein are vectors comprising (i) a polynucleotide sequence encoding a transposon, wherein the transposon comprises: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR. In some embodiments, a vector comprises: (i) a polynucleotide sequence encoding a transposon, wherein the transposon comprises: a 5′ inverted terminal repeat (ITR), a first polynucleotide encoding a gene of interest; and a 3′ inverted terminal repeat (ITR); (ii) a polynucleotide encoding an at least first selection marker located outside of the 5′ ITR and 3′ ITR; and a polynucleotide encoding a transposase located outside of the 5′ ITR and 3′ ITR; wherein the transposase is capable of recognizing the ITRs of the transposon.

In another aspect, provided herein is a set of vectors, comprising: a first vector comprising: (i) a polynucleotide sequence encoding a transposon, wherein the transposon comprises: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR; and (b) a second vector comprising a polynucleotide encoding a transposase, wherein the transposase is capable of recognizing the ITRs of the transposon, wherein upon introducing the set of vectors into a cell the transposon is transposed into the cell's genome and the polynucleotide encoding the selection marker is episomal.

In some embodiments, any of the vectors described herein can be an expression vector. In some embodiments, an expression vector can include one or more promoter sequences (e.g., any of the promoter sequences described herein) operably linked to a coding sequence of any of the proteins capable of modulating cell proliferation described herein. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some embodiments, a vector includes sufficient cis-acting elements that supplement expression where the remaining elements needed for expression can be supplied by the host cell (e.g., the cell line).

In some embodiments, a vector includes a polynucleotide comprising a coding sequence of a first gene of interest. In some embodiments, a vector includes a polynucleotide comprising a coding sequence of a first gene of interest and a coding sequence of a second gene of interest where each are located between the ITR and are transposed into the cell's genome. In such embodiments where the vector includes two or more coding sequences between the ITRs, each of the two or more coding sequences may be operably linked to a promoter sequence or to another coding sequence via a self-cleaving polypeptide or IRES. As used herein, the term “operably linked” is well known in the art and refers to genetic components that are combined such that they carry out their normal functions. For example, a coding sequence is operably linked to a promoter when its transcription is under the control of the promoter. In another example, a coding sequence can be operably linked to other coding sequences by a self-cleaving 2A polypeptide or an internal ribosome entry site (IRES). In such cases, the self-cleaving 2A polypeptide allows the second coding sequence to be under the control of the promoter operably linked to the first coding sequence. In some cases, the coding sequences described herein can be operably linked to any other coding sequence described herein using a self-cleaving 2A polypeptide or IRES.

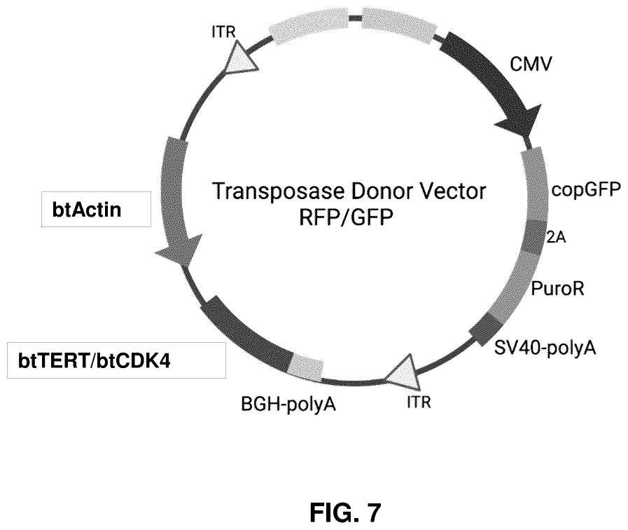

Non-limiting examples of vectors that can be used in the methods described herein are as shown in FIG. 2, and FIG. 7.

In some embodiments, a coding sequence of any polynucleotides described herein is operably linked to a promoter. In some embodiments, the promoter is a tissue-specific promoter. In some embodiments, the tissue-specific promoter is a muscle-specific promoter. In some embodiments, the muscle-specific promoter is selected from the group consisting of: skeletal β-action, myosin light chain 2a, dystrophin, SPc-512, muscle creatine kinase, and synthetic muscle promoters. In some embodiments, the promoter is a constitutively active promoter. In some embodiments, the promoter is selected from the group consisting of: EF1 (e.g., EF1alpha), PGK, CMV, RSV, and β-actin. In some embodiments, the promoter is an EF1 (e.g., EF1alpha) promoter. In some embodiments, the promoter is a PGK promoter.

In some embodiments, the vector comprises a selectable marker. In some embodiments, the selectable marker is an antibiotic resistance protein that confers antibiotic resistance when expressed. In such embodiments, the methods provided herein include a selecting step that comprises contacting the cells with an antibiotic under conditions sufficient to enable selection of the transduced cells.

In some embodiments, the selectable marker is a fluorophore. In such embodiments, the methods provided herein include a selecting step that comprises performing fluorescence activated cell sorting (FACS).

In some embodiments, the selectable marker encodes a nutrient. In such embodiments, the methods provided herein include a selecting step that comprises culturing in a media lacking the nutrient to select from transfected cells

In some embodiments, a vector system is used to integrate a polynucleotide comprising a coding sequence of any of the genes of interested described herein (e.g., any one or more of the proteins capable of modulating cell proliferation), into the genome of a cell line (e.g., any of the cell lines described herein).

### 7.11. Methods of Transducing Cells

Methods of introducing nucleic acids and expression vectors into a cell (e.g., an immortalized cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral, retroviral, and lentiviral transduction), lipid nanoparticle (LNP) transfection, heat shock, nanoparticle transfection, and the like.

### 7.12. Kits

Also provided herein are kits comprising any of the cell lines, any of the cells derived from the cell lines, any of the polynucleotides described herein (e.g., any of the coding sequence of any one or more of the growth factor ligands described herein, any one or more of the growth factor ligands described herein, any of the accessory protein described herein, or a combination thereof). In some embodiments, the kit includes instructions for performing any of the methods described herein.

### 7.13. Cells/Cell Lines

Also provided herein are cell line(s) for cultured food production. In some embodiments, the cell line(s) are capable of self-renewal. In some embodiments, the cell line(s) are immortalized cell line(s). In some embodiments, the cell lines are then differentiated to cell types of interest. The cells are non-human cells, including non-human mammalian cells or avian cells.

Also provided herein are immortalized cells (e.g., any of the immortalized cells described herein). In some embodiments, the immortalized cells are fibroblasts. In some embodiments, the immortalized cells comprise any of the nucleic acids described herein that encode any of the myogenic regulatory factors described herein.

Also provided herein are cells comprising any of the polynucleotides described herein that include any of the genes of interest (e.g., any of the proteins capable of modulating cell proliferation described herein).

Also provided herein are cells that include at least one integrase recognition site integrated into the genome. In some embodiments, the method includes integrating at least a first integrase recognition site prior to integrating the polynucleotide encoding TERT and/or prior to integrating the polynucleotide encoding a protein capable of modulating cell proliferation. Non-limiting examples of methods that can be used to insert the integrase recognition site into the genome include CRISPR/Cas9-mediated knockin, viral-mediated integration (e.g., lentivirus or retrovirus), non-viral mediated integration, Zinc-finger-mediated knockin, and TALE-mediated knockin.

Provided herein, in some embodiments, are cell lines derived from one or more cells having only a single genetic amendment event. For example, the cells may be transfected to integrate a gene of interest and to only episomally transcribe a selection marker. Such instances eliminate the need for a second genetic amendment event typically needed to excise integrated selection genes. Non-integration of common selection markers eliminates potential regulatory concerns uniquely associated with cells that are destined for consumption by humans or non-humans. For instance, cells having integrated selection markers, or even remnants of selection markers, may be designated as unfit for consumption by agencies regulating food production.

Also provided herein are cells derived from the cell line(s). Non-limiting examples of cells derived from the immortalized cells (e.g., using the methods described herein) include myoblasts, myotubes, multinucleated myotubes, satellite cells, skeletal muscle fibers, or any combination thereof.

In some embodiments, the cell line is from a livestock, poultry, game or aquatic animal species. In some embodiments, the cell line or immortalized cell line are from a chicken, duck, or turkey. In some embodiments, the cell line or immortalized cell line are from a fish. In some embodiments, the cell line or immortalized cell line are from a livestock species. In some embodiments, the livestock species is porcine or bovine.

In some embodiments, the cell line is selected from any metazoan species. In some embodiments, the cell line is from any animal, except for human, such as non-human vertebrate and invertebrate animal species. In some embodiments, the cell line is from any vertebrate animal species such as non-human mammals, reptiles, birds, amphibians, and the like. In some embodiments, the cell line is from any mammalian species such as a murine, bovine, porcine, and the like or is an avian species, such as poultry and the like. In some embodiments, the cell line is derived from a species selected from including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, the cell line (e.g., a cell line that is ultimately immortalized) is isolated from Gallus gallus (chicken). In some embodiments, the cell is isolated from chicken skin. In some embodiments, the cell is isolated from chicken muscle. In some embodiment, the cell is isolated from a chicken (e.g., chicken skin or chicken muscle) and cultured until a monoculture of cells is established (e.g., a monoculture of fibroblasts originating from the isolated chicken cells).

In some embodiments, the cell line (e.g., a cell line that is ultimately immortalized) is selected from the group consisting of: a myoblast, an immortalized myoblast, an immortalized primary myoblast, a muscle satellite cell, and a muscle stem cell. In some embodiments, the immortalized cell is an immortalized myoblast or an immortalized primary myoblast.

In some embodiments, the cell line (e.g., a cell line that is ultimately immortalized) is a fibroblast. For example, the cell is an immortalized fibroblast.

In some embodiments, skeletal muscle satellite cells are isolated from a chicken. In adults these are quiescent mononucleated myogenic cells that act as a reserve population of cells, able to proliferate and/or differentiate upon stimulation and give rise to regenerated muscle and to more satellite cells.

In some embodiments, an immortalized cell is not a stem cell (e.g., a muscle stem cell or a muscle satellite cell). In some embodiments, an immortalized cell is not a pluripotent stem cell (e.g., an embryonic stem cell or an induced pluripotent stem cell).

Also provided herein are cell banks comprising immortalized cell lines (e.g., immortalized fibroblast cells lines) generated according to the methods described herein.

In some embodiments, the population of cells are selected from fibroblasts, myofibroblasts, and myogenic cells, or a combination thereof.

In some embodiments, the cells are not natively myogenic (e.g., are non-myogenic cells such as fibroblasts or non-myogenic stem cells that are cultured to become myogenic cells (e.g., in suspension culture or in the cultivation infrastructure)).

In some embodiments, the cells are non-myogenic, and such non-myogenic cells can be programmed to be myogenic, for example the cells may comprise fibroblasts modified to express one or more myogenic transcription factors. In some embodiments, the myogenic transcription factors include MYOD1, MYOG, MYF5, MYF6, PAX3, PAX7, paralogs, orthologs, and genetic variants thereof. In some embodiments, the cells are modified to express one or more myogenic transcription factors as described in a PCT publication, WO/2015/066377, which is herein incorporated by reference in its entirety.

In some embodiments, the cells are genetically modified to inhibit a pathway, e.g. the HIPPO signaling pathway. Exemplary methods to inhibit the HIPPO signaling pathway as described in a PCT Application No. PCT/US2018/031276, which is herein incorporated by reference in its entirety.

In some embodiments, the cells are modified to express telomerase reverse transcriptase (TERT) and/or inhibit cyclin-dependent kinase inhibitors (CKI). In some embodiments, the cells are modified to express TERT and/or inhibit cyclin-dependent kinase inhibitors as described in a PCT publication, WO 2017/124100, which is herein incorporated by reference in its entirety.

In some embodiments, the cells are modified to express glutamine synthetase (GS), insulin-like growth factor (IGF), and/or albumin. Exemplary methods of modifying cells to express GS, IGF, and/or albumin are described in a PCT Application No. PCT/US2018/042187 which is herein incorporated by reference in its entirety.

In some embodiments, the cell-based meat product has various characteristics. Exemplary characteristics of the cell-based meat are described in U.S. application Ser. No. 17/033,635 and PCT Application No. PCT/US2021/016681, which are herein incorporated by reference in their entireties.

In some embodiments the cells are genetically edited, modified, or adapted to grow without the need of specific ingredients including specific amino acids, carbohydrates, vitamins, inorganic salts, trace metals, TCA cycle intermediates, lipids, fatty acids, supplementary compounds, growth factors, adhesion proteins and recombinant proteins.

In some embodiments, the cells may comprise any combinations of the modifications described herein.

### 8. ADDITIONAL EMBODIMENTS

Embodiment 1. A transposon/transposase system for transposing and selecting of transposed cells, the system comprising: (a) a transposase or polynucleotide encoding a transposase; and (b) an exogenous nucleic acid sequence comprising: (i) a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, wherein the transposase is capable of recognizing the ITRs of the transposon, wherein upon introducing (a) and (b) into a cell the transposon is transposed into the cell's genome and the polynucleotide encoding the selection marker is episomal.

Embodiment 2. A transposon/transposase system for transposing and selecting of transposed cells, the system comprising: (a) a first vector comprising a polynucleotide encoding a transposase; and (b) a second vector comprising: (i) a transposon comprising: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR, wherein the transposase is capable of recognizing the ITRs of the transposon, wherein upon introducing (a) and (b) into a cell the transposon is transposed into the cell's genome and the polynucleotide encoding the selection marker is episomal.

Embodiment 3. A vector for transposing and selecting of transposed cells comprising: (a) a polynucleotide sequence encoding a transposon comprising: a 5′ inverted terminal repeat (ITR), a first polynucleotide encoding a gene of interest; and a 3′ inverted terminal repeat (ITR); and (b) a polynucleotide encoding an at least first selection marker located outside of the 5′ ITR and 3′ ITR; and (c) a polynucleotide encoding a transposase located outside of the 5′ ITR and 3′ ITR; wherein the transposase is capable of recognizing the ITRs of the transposon, wherein upon introducing the vector into a cell the transposon is transposed into the cell's genome and the polynucleotide encoding the selection marker is episomal.

Embodiment 4. A set of vectors for transposing and selecting of transposed cells comprising: (a) a first vector comprising: (i) a polynucleotide sequence encoding a transposon, wherein the transposon comprises: a 5′ inverted terminal repeat (ITR), a first polynucleotide encoding a gene of interest; and a 3′ inverted terminal repeat (ITR); and (ii) a polynucleotide encoding an at least first selection marker located outside of the 5′ ITR and 3′ ITR; and (b) a second vector comprising a polynucleotide encoding a transposase, wherein the transposase is capable of recognizing the ITRs of the transposon, wherein upon introducing the set of vectors into a cell the transposon is transposed into the cell's genome and the polynucleotide encoding the selection marker is episomal.

Embodiment 5. The system, vector, or set of vectors of any one of embodiments 1-4, wherein the transposase mediating transposition of the transposon is selected from Sleeping Beauty, PiggyBac, Frog Prince, Himarl, Passport, Minos, hAT, Tol1, Tol2, AciDs, PIF, Harbinger, Harbingert-D, and Hsmarl, or a functional fragment thereof.

Embodiment 6. The system, vector, or set of vectors of any one of embodiments 1-5, wherein the first polynucleotide encoding the gene of interest is operably linked to a first promoter.

Embodiment 7. The system, vector, or set of vectors of any one of embodiments 1-6, wherein the transposon comprises a polynucleotide encoding a second gene of interest.

Embodiment 8. The system, vector, or set of vectors of embodiment 7, wherein the polynucleotide encoding the second gene of interest to the first gene of interest or a second expression control element.

Embodiment 9. The system, vector, or set of vectors of embodiment 8, wherein the polynucleotide encoding the second gene of interest is positioned between the 5′ ITR and 3′ITR such that the second gene of interest is transposed into the cellular genome.

Embodiment 10. The system, vector, or set of vectors of any one of embodiments 1-7, wherein the gene of interest, the second gene of interest, or both, are selected from: a gene capable of modulating cell proliferation, a growth factor ligand, a growth factor receptor, a myogenic transcription factor, or a combination thereof.

Embodiment 11. The system, vector, or set of vectors of any one of embodiments 1-10, wherein the gene of interest, the second gene of interest, or both, are genes capable of modulating cell proliferation.

Embodiment 12. The system, vector, or set of vectors of embodiment 11, wherein the gene capable of modulating cell proliferation is selected from TERT, BMI-1, CDK4, mutant CDK4, Cyclin D1, PCG1α, Nanog, DCK1, and YAP.

Embodiment 13. The system, vector, or set of vectors of any one of embodiments 7-12, wherein the second gene of interest is a second selection marker.

Embodiment 14. The system, vector, or set of vectors of any one of embodiments 1-13, wherein the first selection marker, the selection marker, or both, are selected from: an antibiotic resistance gene, a fluorophore, or a cell surface marker.

Embodiment 15. The system, vector, or set of vectors of embodiment 14, wherein the polynucleotide encoding an at least first selection marker is operably linked to a second promoter.

Embodiment 16. The system, vector, or set of vectors of any one of embodiments 1-15, wherein the 5′ ITR, 3′ ITR, or both comprise one or more modifications that accelerate transposase activity.

Embodiment 17. The system, vector, or set of vectors of any one of embodiments 1-16, wherein the 5′ ITR has a nucleotide sequence of SEQ ID NO: 17 and the 3′ ITR has a nucleotide sequence of SEQ ID NO: 18.

Embodiment 18. The system, vector, or set of vectors of any one of embodiments 1-17, wherein, upon being introduced into the cell and following transposition, the episomal polynucleotide encoding the first selection marker remains in the cell for at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

Embodiment 19. The system, vector, or set of vectors of any one of embodiments 1-17, wherein, upon being introduced into the cell and following transposition, the episomal polynucleotide encoding the first selection marker remains in the cell for at least 5 days, at least 6 days, at least 7 days, at least 8 days, at least 9 days, at least 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

Embodiment 20. A cell comprising the system, vector, or set of vectors of any one of the preceding embodiments.

Embodiment 21. A method for generating a cell line suitable for dietary consumption, the method comprising: (a) introducing into a population of cells a transposon/transposase system of any one of embodiments 1-18; (b) selecting step (a) transduced cell from the population of non-human cells using the first selection marker; and (c) culturing the transduced cells under condition sufficient to expand the transduced cells.

Embodiment 22. A method for generating a cell line suitable for dietary consumption, the method comprising: (a) introducing into a population of non-human cells a vector or set of vectors of any one of embodiments 3-18; (b) selecting a transduced cell from the population of cells using the first selection marker; and (c) culturing the transduced cells under condition sufficient to expand the transduced cells.

Embodiment 23. The method of embodiment 21 or 22, further comprising a second selecting step.

Embodiment 24. The method of embodiment 23, wherein the second selecting step comprises using the second selection marker to further select the transduced cell.

Embodiment 25. The method of any one of embodiments 21-24, wherein the second selecting step is performed 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the first selecting step.

Embodiment 26. The method of any one of embodiments 21-25, wherein the episomal polynucleotide encoding the selection marker remains in the cell following introduction for at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

Embodiment 27. The method of any one of embodiments 21-26, wherein prior to introducing (or incorporating) the gene of interest encoding a protein that is capable of modulating cell proliferation, the population of bovine cells have a population doubling level (PDL) of no more than 54.

Embodiment 28. The method of any one of embodiments 21-27, wherein, after introducing (or incorporating) the gene of interest encoding a protein that is capable of modulating cell proliferation, the population of bovine cells have a population doubling level (PDL) of at least 55.

Embodiment 29. The method of any one of embodiments 21-28, wherein the cell line is derived from: livestock, poultry, or game animal species.

Embodiment 30. The method of embodiment 29, wherein the cell line is derived from: chicken, duck, or turkey.

Embodiment 31. The method of embodiment 29, wherein the cell line is derived from livestock species.

Embodiment 32. The method of embodiment 31, wherein the livestock species is bovine or porcine.

Embodiment 33. The method of any one of embodiments 21-32, wherein the wherein the bovine cells are non-myogenic cells.

Embodiment 34. The method of embodiment 33, wherein the non-myogenic cells are selected from: fibroblasts, mesenchymal cells, and chondrocytes.

Embodiment 35. The method of any one of embodiments 21-32, wherein the bovine cells are myogenic cells.

Embodiment 36. The method of embodiment 35, wherein the myogenic cells are myoblasts, myocytes, satellite cells, side population cells, muscle derived stem cells, myofibroblasts, mesenchymal stem cells, myogenic pericytes, or mesoangioblasts.

Embodiment 37. A cell-based meat product produced using the method of any one of embodiments 21-36.

Embodiment 38. A population of cells suitable for consumption produced by any of the methods of embodiments 21-36.

Embodiment 39. A population of immortalized bovine cells suitable for consumption produced by any of the methods of embodiments 21-36.

Embodiment 40. A cell comprising: a genome having an integrated polynucleotide encoding a gene of interest between a 5′ ITR and a 3′ ITR, wherein the 5′ ITR and the 3′ ITR is transposable by a transpose; and an episomal polynucleotide encoding a selection marker.

Embodiment 41. The cell of embodiment 40, wherein the gene of interest is selected from genes capable of modulating cell proliferation, a growth factor ligand, a growth factor receptor, a myogenic transcription factor, or a combination thereof.

Embodiment 42. The cell of embodiment 41, wherein the gene of interest is a gene capable of modulating cell proliferation.

Embodiment 43. The cell of embodiment 42, wherein the gene capable of modulating cell proliferation is selected from TERT, BMI-1, CDK4, mutant CDK4, Cyclin D1, PCG1α, Nanog, DCK1, and YAP.

Embodiment 44. The cell of any one of embodiments 40-43, wherein the selection marker is selected from: an antibiotic resistance gene, a fluorophore, or a cell surface marker.

### 9. EXAMPLES

### 9.1. Experimental Procedures/Methods

### 9.1.1. Integration of Gene of Interest

In one example, cells are transfected with 2 plasmids. One plasmid contains the transposase, and the second plasmid contains the transposon having the gene of interest. Transposase and transposon are transfected at a 1:1 ratio to 4:1 ratio with a transfection reagent that is most compatible to the cell line. Once transfected, the selection marker (e.g. GFP expression) is confirmed after 48 hours and transfected cells are enriched via flow cytometer. This cell population is recovered and expanded. The fluorescence GFP signature is measured via flow cytometry. Cells are then collected at designated time points to extract genomic DNA. The backbone plasmid DNA and the GOI DNA is confirmed via ddPCR.

### 9.1.2. Assessment of Cell Proliferation

In some examples, adherent cells are seeded at 5,000-10,000 cells/cm2 with cell culture media. Cells are grown and expanded for about 2-4 days until cells reach 70-90% confluency. Once sufficient confluence is reached, the media is aspirated and cells are washed with a phosphate saline buffer. The phosphate saline buffer is aspirated, and trypsin-EDTA is added to the vessel and incubated for 3-10 minutes at 25-37 C. Detached single cells are collected and counted for the total cell count harvested from one vessel. Once counted, cells are seeded in a fresh vessel at the designated cell density mentioned above. Cell growth is measured by the output of total cells relative to the input of total cells at a given amount of time.

$$PDLi = Previously calculated PDL$$

$$PDLf = final calculated PDL$$

$$Yi = initial population of cells$$

$$Yf = final population of cells$$

$$DT = doubling time$$

$$t = time in culture ( hours )$$

$$PD = population doublings that occured in culture period to t hours$$

$$PD = ( LN ( Yf / Yi ) / LN ( 2 ) )$$

$$DT = t / PD$$

$$PDLf = PDLi + PD$$

### 9.2. Assessment of Myogenicity

Myogenicity can be assessed using qRT-PCR (real-time quantitative reverse transcription). mRNA is isolated from cells to examine gene expression with probes specifically designed to amplify select target genes to characterize cell lines. Identical quantity of mRNA is reverse transcribed to generate cDNA. Each cDNA is submitted to quantitative PCR (qPCR) to assess the expression of myogenic factors relative to a housekeeping gene. Expression of MyoD, MyoG, and/or MyHCle indicate myogenic cells. Additionally, high levels of MyHCle are indicative of cells that can mature to form myotubes.

Myogenicity can be assessed using immunohistochemistry. Cells are seeded in a 96-well plate at a low density (5000-10,000 cells/cm2) to allow cells to grow in the presence or absence of different small molecule combinations. After 2 days of media exposure, cells are fixed with 4% paraformaldehyde (PFA) and washed. Cells are permeabilized with 0.05% PBS-T (triton-x), blocked with normal goat serum (source) and are incubated with antibodies, and subsequently with secondary antibodies.

### 9.3. Example 1: Experimental Design for Assessing the Transposon System

This experiment was designed to assess feasibility of using the transposon systems described in FIG. 1 and FIG. 2 to generate a cell line comprising cells with a gene of interest integrated into the cellular genome. For example, FIG. 1 shows a schematic for transposition of the transposon comprising a promoter and a gene of interest into a cellular genome. The Inverted terminal repeats (ITR) flank the transposable element (TE) and are not actually part of the transposable element. The ITRs play a role in insertion of the TE. Moreover, after a TE is excised, these repeats are left behind as “footprints.” Sometimes, these footprints alter gene expression (i.e., expression of the gene in which they have been left behind) even after their related TE has moved to another location on the genome. In step 2 of FIG. 1, a transposase that can recognize the ITRs of the transposon in introduced into the cell under conditions such that the transposon is transposed into the cellular genome and the polynucleotide encoding the selection marker is episomal following transposition. By residing episomally, the polynucleotide encoding the selection marker can be used to select the transduced cells, because transcription still occurs episomally—at least temporarily, without actually being integrated into the cellular genome (see FIG. 3). The episomal polynucleotide encoding the selection marker is eventually degraded or eliminated from the cell.

### 9.4. Example 2: Assessment of the/Transposon System in Chicken Cells

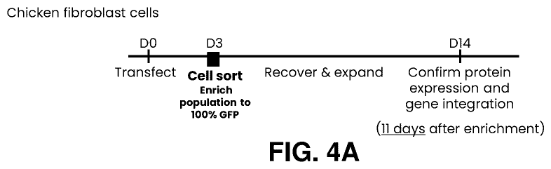

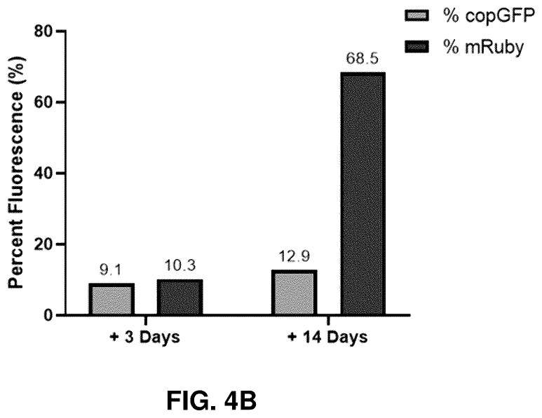

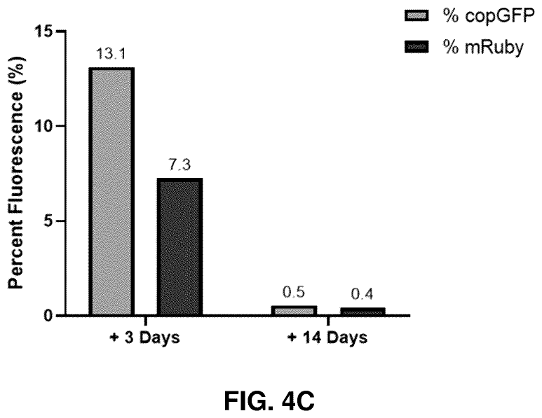

In this experiment, the transposon/transposase system as described in Example 1 was assessed in suspension and serum free adapted chicken fibroblast cells. FIG. 4A shows an exemplary workflow for the experiments performed in this Example.

In particular, chicken fibroblast cells were transfected with 0.6 μg transposon donor plasmid (PL 562)+/−2.4 μg transposase plasmid (PL573) using lipofection. Transfection efficiency was quantified via FACS 48-72 h post transfection. 48-72 h after transfection, cells containing the plasmid (GFP+ and/or mRuby+) were enriched via cell sorting and cultured. FIG. 4B shows percent fluorescence of for copGFP and mRuby when cells are transfected with Donor Vector with the transposase-which integrates mRuby and episomally delivers copGFP—at 3 days post transfection and again at 14 days post transfection. FIG. 4C shows percent fluorescence of for copGFP and mRuby when cells are transfected with Donor Vector without transposase at 3 days post transfection and again at 14 days post transfection. Without transposase, the Donor Vector is completely lost at 14 days post transfection. Overall, this data showed that the transposon/transposase system provided herein could be used to integrate back bone free GOI.

### 9.5. Example 3: Assessment of the Transposon System in Bovine and Chicken Cells

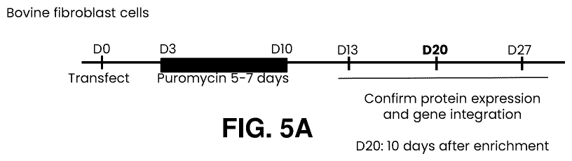

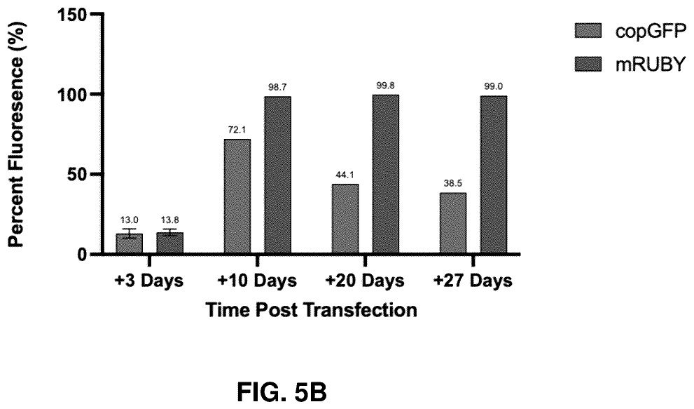

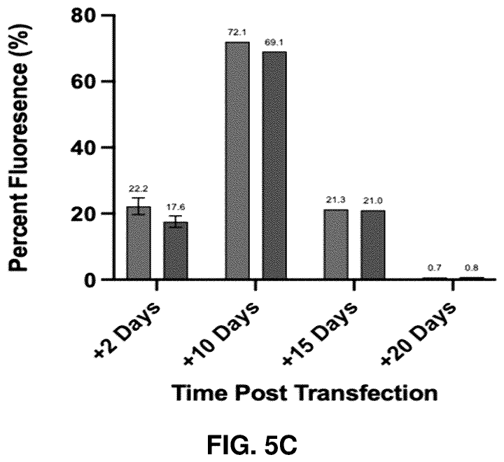

In this experiment, the transposon/transposase system as described in Example 1 was assessed in bovine fibroblasts cells. FIG. 5A shows an exemplary workflow for the experiments performed in this Example.

In this example, the transposon/transposase system was assessed for its ability to deliver a backbone free gene of interest in a bovine cell line, i.e. delivering a gene of interest without integrating selection marker backbone.

FIG. 5A shows a non-limiting experimental workflow used in Example 2. In particular, bovine cells (“4C-TCC”) were transfected with 0.6 μg transposon donor plasmid (PL 562)+/−2.4 μg transposase plasmid (PL573) using lipofection. Transfection efficiency was quantified via flow cytometry 48-72 h post transfection. 72 h after transfection cells containing plasmid were enriched via puromycin selection for 7 days. GFP and RFP expression was routinely quantified via flow cytometry at each passage. n=1-2+/−SEM.

FIG. 5B shows percent fluorescent for copGFP and mRUBY for bovine cells transfected with Donor Vector and transposase that integrate mRuby and episomally deliver copGFP. FIG. 5C shows percent fluorescence of for copGFP and mRuby when cells are transfected with Donor Vector without transposase at 2 days post transfection and again at 10, 15, 20 days post transfection. Without transposase, the Donor Vector is completely lost at 20 days post transfection. Overall, this data showed that the transposon/transposase system provided herein could be used to integrate back bone free GOI.

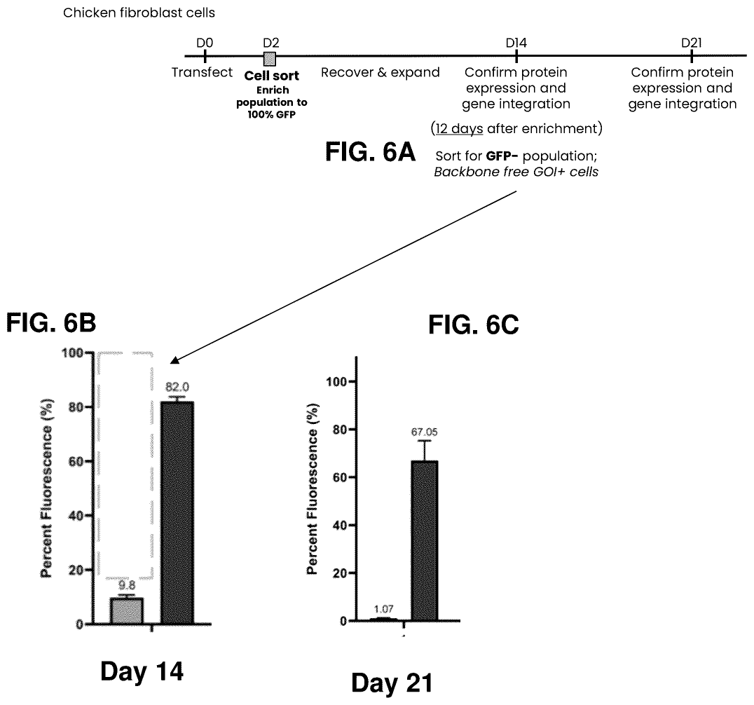

FIG. 6A shows a non-limiting experimental workflow used in Example 2._In particular, suspension and serum free adapted chicken fibroblast cells were transfected with 0.6 μg donor plasmid (PL 562)+/−2.4 μg transposase plasmid (PL573) using lipofection. Transfection efficiency was quantified via flow cytometry 48 h post transfection to enrich GFP population. GFP and RFP expression was routinely quantified via flow cytometry at each passage. n=1-2+/−SEM

FIG. 6B shows percent fluorescent for copGFP and mRUBY for bovine cells transfected with Donor Vector and transposase at 14 days post transfection and was enriched by the GFP—population. FIG. 6C shows percent fluorescent for copGFP and mRUBY for bovine cells transfected with Donor Vector and transposase at 21 days post transfection. By selecting out GFP—population, this generated a backbone free population as a proof of concept.

FIG. 7 is a schematic of a donor vector that includes TERT and CDK4 genes inside the ITR sequences of the transposon and copGFP and PuroR selection markers outside the ITR sequences.

### 9.6. Example 4: Generation of Bovine Cell Lines Using the Transposase/Transposon Systems

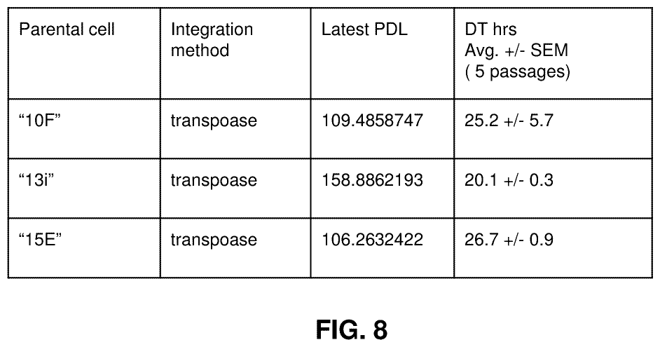

In the examples provided by FIGS. 8-10, the transposon/transposase system was used to generate bovine cell lines. Cell lines that were transfected with the transposon system include: “9B” and “13I” Genes to be introduced using the transposase systems include: TERT-CDK4 (bovine codon optimized) driven by a btActinB promoter.

FIG. 8 provides a PDL count and doubling time average across three cell lines, each having different parental cells and each with genes of interest integrated via transposase and episomally expressed selection markers. In one embodiment, progeny from parental cell line 10F (1° F. cell line) were transfected via transposase to integrate TERT/CDK4 (shortened to “T/C”) and to episomally express puromycin resistance gene and GFP. The cell lines were initially selected via episomally expressed selection markers. The 10F cell line was passaged to a population doubling level (PDL) of 109, while retaining proliferative capacity. The 10F cell line maintained a doubling time average, calculated across 5 passages, of 25.2 hours plus or minus 5.7 hours. In another embodiment, progeny from parental cell line 13i (13i cell line) were transfected via transposase to integrate T/C. The 13i cell line was passaged to a population doubling level (PDL) of 159, while retaining proliferative capacity. The 13i cell line maintained a doubling time average, calculated across 5 passages, of 20.1 hours plus or minus 0.3 hours. In another embodiment, progeny from parental cell line 15E (15E cell line) were transfected via transposase to integrate T/C. The 15E cell line was passaged to a population doubling level (PDL) of 106, while retaining proliferative capacity. The 15E cell line maintained a doubling time average, calculated across 5 passages, of 26.7 hours plus or minus 0.9 hours.

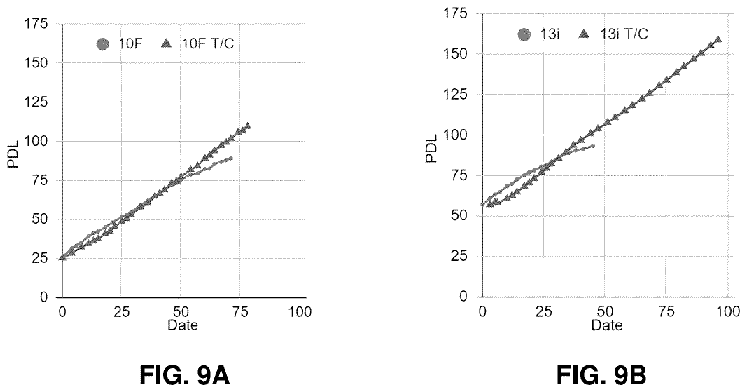

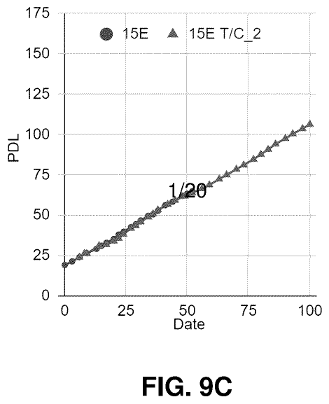

FIG. 9A-9C show the results for the bovine cell lines transfected with transposase genes to generate immortalized cell lines. Transfection occurred at different PDLs for each cell line. Specifically, PDL of control cell lines 10F, 13I, and 15E (data points illustrated as dots) are compared to transfected cell lines 10F T/C, 13I T/C, and 15E T/C (data points illustrated as triangles). All controls died before PDL 100, indicating complete senescence. In contrast, the transfected cell lines all survived beyond PDL 100. This indicates that integrative T/C transfection and transient selection marker transfection extends replicative capacity of all three bovine cell lines tested while simultaneously eliminating the need to remove the backbone, which, due to its transient nature, automatically degrades over a short time frame. FIG. 9A shows population doubling levels (PDL) for the 10F bovine cell line transfected with either a transposon system that integrated T/C or wild type non-transfected control. FIG. 9B shows PDL for the 13i bovine cell line transfected with the transposon system that integrated T/C or wild type non-transfected control. FIG. 9C shows PDL for the 15E bovine cell line transfected with the T/C transposon system or wild type control.

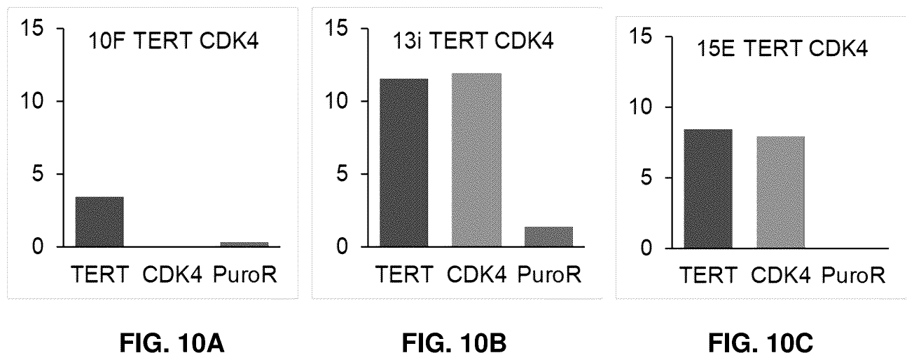

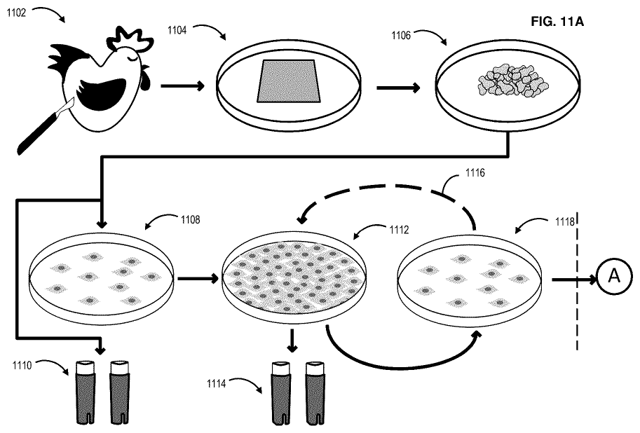

FIGS. 10A-10C show copy number quantification of TERT, CDK4 or PuroR (plasmid backbone component) genes for cell lines 10F, 13i, and 15E 12 to 15 PDLs after they have been transfected by a transposon system, which integrates T/C and transiently expresses PuroR. This data supports that cells transfected with this transposon system enable selection of cells having the gene of interest, which is indicated by the relatively high copy number of the integrated genes, while simultaneously having minimal to zero expression of the transiently expressed selection marker after 12 to 15 PDLs, which indicates the transiently expressed sequence's transient nature. FIG. 10A shows copy numbers of the 10F T/C cell line. In this example, 0.3 copies of puromycin backbone fraction remains, and this quantity is expected to reach zero with further passaging, while TERT is integrated. FIG. 10B shows copy numbers of 13i T/C cell line. In this example, 1.5 copies of puromycin backbone fraction remains, and this quantity is expected to hit zero with further passaging, while TERT and CDK4 are integrated. FIG. 10C shows copy numbers of 15E T/C cell line. In this example, 0.06 copies of puromycin backbone fraction remains, and this quantity is expected to hit zero with further passaging, while TERT and CDK4 are integrated. This data thereby provides proof that a majority of cells transfected with the transposon system stably express the gene of interest, while only a small minority express the selection marker. This advantageously enables clonal isolation of a cell line having the gene of interest integrated and lacking the selection marker, while eliminating the need for further genetic modification that is typically required to remove integrated selection marker backbones. As a further elaboration, even if the transient selection marker exists in a small subset of the population, obtaining a clone without it is quite easy, and that clone can be used to start a cell line for the production of cultivated meat.

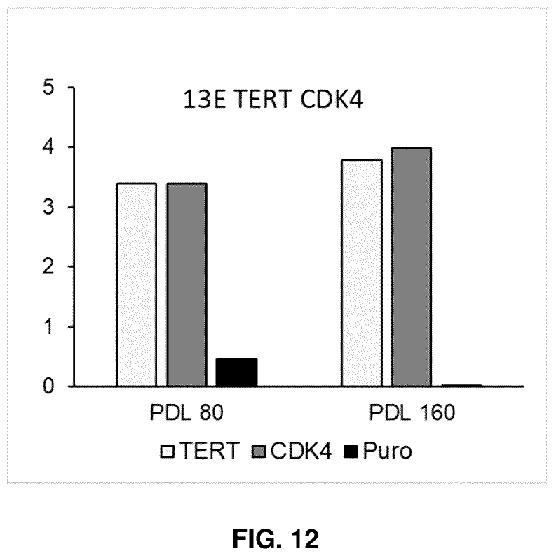

FIG. 12 shows copy number quantification of TERT, CDK4, and Puromycin in a 13E cell line at 80 PDL and 160 PDL after introduction of a transposon vector system as described herein with a transposon construct comprising the TERT, CDK4 and PuroR coding sequences, with the TERT and CDK4 sequences inside the 5′ ITR and 3′ ITR sequences and the PuroR coding sequence outside the 5′ ITR and 3′ITR sequences (see, FIG. 7). Specifically, the 13E cell line at PDL 80 (having 32 population doublings since transfection) has 3.5 copies of TERT and CDK4 and 0.4 copies of PuromycinR backbone. Once the 13E cell line achieved 160 PDL (having 112 population doublings since transfection) it retained 4 copies of TERT and CDK4 and no detectable copies of PuromycinR sequences. This data support that the PuromycinR portion of the transfected plasmid was only transiently maintained, while the TERT and CDK4 portions of the transfected plasmid were stability integrated. Of note, during the 13E cell lines expansion from PDL 80 to PDL 160, the 13E cells were transitioned from serum containing adherent culture to an animal component free suspension culture, thereby showcasing the robustness and commercial viability of this transfection method.

### 9.7. Example 5: Procurement and Growth of Cells into a Cell Mass Suitable for Consumption

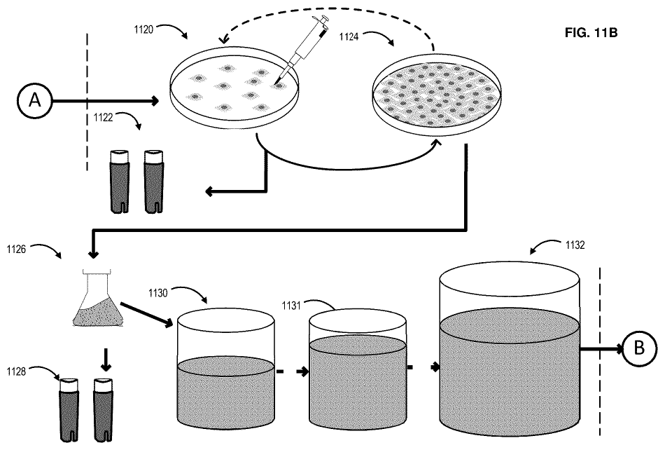

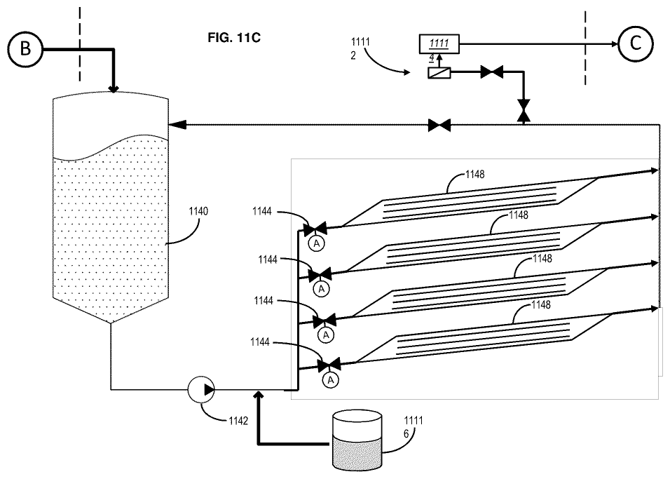

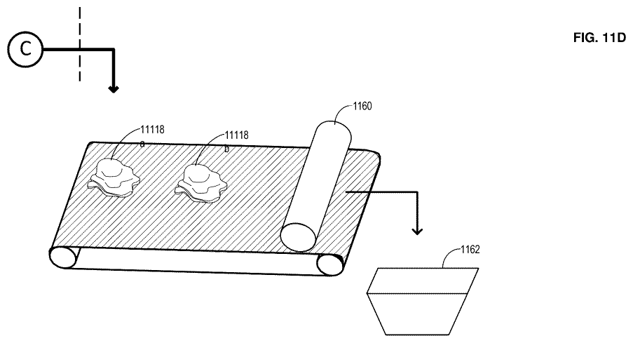

FIGS. 11A-11D and the following accompanying paragraphs describe procurement of cells and growth of cells into a cell mass in accordance with one or more embodiments. Generally, FIGS. 11A-11D illustrate a process of collecting cells from an animal, growing cells in a favorable environment, banking successful cells, and collecting cells into a cell mass followed by de-wetting and/or other treatments.

As illustrated by step 1102 in FIG. 11A, tissue is collected from a living animal via biopsy. In particular, stem cells, mesenchymal progeny, ectoderm lineage, and/or endoderm lineages can be isolated from the removed tissue. In some implementations of the present disclosure, tissue, such as fat and others, are processed to isolate stem cells, mesenchymal, ectoderm, and/or endoderm progeny or lineage cells. As illustrated, tissue 1104 is removed from an animal. In some examples, the tissue 1104 is removed from a living animal by taking a skin sample from the living animal. For instance, skin or muscle samples may be taken from a chicken, cow, fish, shellfish or another animal.

Cells may be extracted from the tissue 1104 that was removed from the animal. More specifically, the tissue 1104 is broken down by enzymatic and/or mechanical means. To illustrate, FIG. 11A includes digested tissue 1106 that comprises the cells to be grown in cultivation.

Cells in the digested tissue 1106 may be proliferated under appropriate conditions to begin a primary culture. As illustrated in FIG. 11A, cells 1108 from the digested tissue 1106 are spread on a surface or substrate and proliferated until they reach confluence. As shown in FIG. 11A, in some cases, cells 1112 have reached confluence when they start contacting other cells in the vessel, and/or have occupied all the available surface or substrate.

In some examples, cells are stored and frozen (i.e., banked) at different steps along the cell culture process. Cryopreservation generally comprises freezing cells for preservation and long-term storage. In some implementations, tissue and/or cells are removed from a surface or substrate, centrifuged to remove moisture content, and treated with a protective agent for cryopreservation. For example, as part of cryopreservation, tissues and cells are stored at temperatures at or below −80° C. The protective agent may comprise dimethyl sulfoxide (DMSO) or glycerol.

Cells stored through cryopreservation may be used to replenish working cell stock. For instance, while a portion of the digested tissue 1106 is used as the cells 1108 spread on a surface or substrate, the remaining or excess digested tissue 1106 is transferred to cryovials 1110 for storage. Furthermore, the cells 1112 may be banked once reaching confluence and stored in cryovials 1114.

Once the cells 1112 have reached confluence, or just before the cells 1112 have reached confluence (e.g., occupation of about 80% of the substrate), the disclosed process comprises a series of cell passage steps. During cell passage, the cells 1112 are divided into one or more new culture vessels for continued proliferation. To illustrate, the cells 1112 may be diluted or spread on one or more surfaces or substrates to form the cells 1118. The cells 1118 are then grown 1116 to confluence, or just before confluence.

The cycle of dividing the cells 1112 into the cells 1118 for continued proliferation in new culture vessels may be repeated for a determined number of cycles. Typically, cell lines derived from primary cultures have a finite life span. Passaging the cells allows cells with the highest growth capacity to predominate. In one example, cells are passaged for five cycles to meet a desired genotypic and phenotypic uniformity in the cell population.

In some implementations, the disclosed method comprises immortalizing cells that have been grown and passaged for the determined number of cycles. For instance, the cells 1118 may be immortalized. As shown in FIG. 11B, cells 1120 have demonstrated a preferred growth capacity to proceed to immortalization. To achieve immortalization, the disclosed process transfects the cells 1120 with genes of interest. In one example telomerase reverse transcriptase (TERT) is introduced to the cells 1120. In some embodiments, the cells may be subjected to a selection process as known by those skilled in the art. The cells 1120 may then be passaged for a predetermined set of passaging cycles. In one example passaging cycle, the cells 1120 are grown to (or near) confluence 1124, then they are reseeded in new growth vessels, preserved in vials 1122, or some combination of both. The disclosed process may include any number of passaging cycles to ensure that the cells have reached immortality (e.g., can passage 60+ times without senescing), a target growth capacity, and/or a target quantity for banking. For example, cells may be passaged until they have reached a passage level of 100 (e.g., have been passaged for 100 passaging cycles). In another example, cells are passaged until they reach a population doubling level of 100.

Cells that have reached immortality or a target growth capacity by living through a target passage level may be adapted to suspension culture. In one example, a suspension culture media and agitation of cells in this suspension environment help cells to adapt and start proliferating in the new growth environment. The cells adapted to suspension 1126 may be stored in cryovials 1128 for cryopreservation and banking. Cells in suspension 1126 will begin to proliferate and the process begins a series of dilute and expand steps.

During dilution and expansion, cells are moved from growth vessels into newer, and progressively larger, growth vessels. For example, cells in suspension 1126 may begin in a single tube. The cells will proliferate and increase in cellular density. Once the cells have reached a target cell number (i.e., viable cell density (VCD) at desired volume), they are diluted and moved to a larger growth vessel. Optionally, the cells are banked in cryovials throughout expansion. For example, once cells in suspension reach a maximum VCD, the cells may begin to leave exponential growth due to overcrowding. After reaching a target density, the suspension cells may be transferred to a larger vessel 1130 and diluted with additional media. The dilute-and-expand steps are repeated using progressively larger vessels (e.g., the vessel 1131 and the vessel 1132) and/or progressive dilution until the cells reach a production-ready volume. For example, cells may be production ready at about a 1,000-100,000 liter scale at 5 million cells per mL. The cells may be banked in cryovials at any of the dilution and expansion cycles.

As part of preparing cells to form cell-based-meat products, the disclosed process comprises growing the cells as an adherent culture. Generally, cells that are grown attached to a substrate form a texture that more closely resembles tissue found in conventional meat. Thus, the cells may be transferred from growth in suspension to growth in an adherent reactor. For example, the cells grown in suspension in the vessel 1132 may be transferred to growth on a substrate. FIG. 11C illustrates a bioreactor system comprising a plurality of adherent bioreactors 1148 connecting in parallel to a media vessel 1140. The media vessel 1140 holds the cells grown in suspension media. In some implementations, cells from the vessel 1132 are transferred directly to a cell culture media (or just “media”) vessel 1140. In one example, the media vessel 1140 comprises the vessel 1132. The adherent bioreactors 1148 may comprise pipe-based bioreactors. As shown, a plurality of valves 1144 is secured to the plurality of adherent bioreactors 1148 to enable individual use and access of each of the adherent bioreactors 1148. For instance, to limit flow to only a first bioreactor of the plurality of adherent bioreactors 1148, the valve 1144 of the first bioreactor is opened while the remaining valves 1144 are closed. Furthermore, the bioreactor system can include a directional valve 1142 for changing between flow directions.

In some implementations, and as illustrated in FIG. 11C, cells (e.g., adherent cells or suspension adapted cells) are prepared by flowing cells suspended in media (e.g., cell culture media) across substrates in the plurality of adherent bioreactors 1148. More particularly, cells from the media vessel 1140 may contact or land on the substrates in the plurality of adherent bioreactors 1148. Cells and media that flowed through the adherent bioreactors 1148 are cycled back to the media vessel 1140. The media and cells can be cycled through the adherent bioreactors 1148 until a target adherent cell density is reached. For instance, in some implementations, the disclosed method comprises measuring a cell density of outflow from the adherent bioreactors 1148 to infer an adherent cell density.

The cells grow into adherent tissue within the adherent bioreactors 1148. Once they have grown to a target density, either according to a learned timing or according to a measured fluctuation in cell metabolism of components such as glucose and oxygen, then the adherent tissue is ready for removal. The removal process of the disclosed method uses a high-pressure flow to shear the adherent tissue off the substrate surfaces. In one example, wash buffer from a wash tank 1156 is flowed across the substrates in the adherent bioreactors 1148. The wash buffer and cell mixture are flowed through a filter 1152 where the cells are collected into one or more cell masses 1154.

The cell masses 1154 may be further processed to adjust moisture content. FIG. 11D illustrates an example apparatus for reducing moisture content in the cells. In particular, FIG. 11D illustrates a pressure apparatus 1160 that compresses the cell masses 1158a and 1158b. While FIG. 11D illustrates a mechanical method for adjusting moisture content of the cell masses 1158a and 1158b, other methods may be used to adjust moisture content. For example, the cell masses 1158a and 1158b may be mixed with a drying agent, vacuum dried, centrifuged, or otherwise dried. A moisture-adjusted-cell mass may be transferred to a container 1162 for additional processing. For example, the cell mass 1158a or 1158b may be removed from the container 1162 to be formed into a cell-based-meat product.

### 10. Equivalents and Incorporation by Reference

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

SEQUENCE APPENDIX

| SEQ ID | | |
|---|---|---|
| NO: | Description | Sequence |
| 1 | Bos taurus | MPRAPRCRAVRALLRASYRQVLPLAAFVRRLRPQGHRLVRRGDPAAFRALVAQ |
| | TERT | CLVCVPWDAQPPPAAPSFRQVSCLKELVARVVQRLCERGARNVLAFGFTLLAG |
| | Amino acid | ARGGPPVAFTTSVRSYLPNTVTDTLRGSGAWGLLLHRVGDDVLTHLLSRCALY |
| | | LLVPPTCAYQVCGPPLYDLRAAAAAARRPTRQVGGTRAGFGLPRPASSNGGHG |
| | | EAEGLLEARAQGARRRRSSARGRLPPAKRPRRGLEPGRDLEGQVARSPPRVVT |
| | | PTRDAAEAKSRKGDVPGPCRLFPGGERGVGSASWRLSPSEGEPGAGACAETKR |
| | | FLYCSGGGEQLRRSFLLCSLPPSLAGARTLVETIFLDSKPGPPGAPRRPRRLP |
| | | ARYWQMRPLFRKLLGNHARSPYGALLRAHCPLPASAPRAGPDHQKCPGVGGCP |
| | | SERPAAAPEGEANSGRLVQLLRQHSSPWQVYGLLRACLRRLVPAGLWGSRHNE |
| | | RRFLRNVKKLLSLGKHGRLSQQELTWKMKVQDCAWLRASPGARCVPAAEHRQR |
| | | EAVLGRFLHWLMGAYVVELLRSFFYVTETTFQKNRLFFFRKRIWSQLQRLGVR |
| | | QHLDRVRLRELSEAEVRQHQEARPALLTSRLRFVPKPGGLRPIVNVGCVEGAP |
| | | APPRDKKVQHLSSRVKTLFAVLNYERARRPGLLGASVLGMDDIHRA |
| 2 | Bos taurus | ATGCCGCGCGCGCCCAGGTGCCGGGCCGTGCGCGCCCTTCTGCGGGCCAGCTA |
| | TERT | CCGGCAGGTGCTGCCCCTGGCCGCCTTCGTACGGCGCCTGCGGCCCCAGGGCC |
| | Nucleic acid | ACCGGCTTGTGCGGCGCGGGGACCCGGCGGCCTTCCGCGCGCTGGTGGCTCAG |
| | | TGCTTGGTGTGCGTGCCCTGGGACGCGCAGCCGCCCCCTGCCGCCCCGTCCTT |
| | | CCGCCAGGTGTCCTGCCTGAAGGAGCTGGTGGCCAGAGTCGTGCAGAGGCTCT |
| | | GCGAGCGCGGCGCGAGGAACGTGCTGGCCTTCGGCTTCACGCTGCTGGCCGGG |
| | | GCCCGCGGCGGGCCGCCCGTGGCCTTCACGACCAGCGTACGCAGCTACCTGCC |
| | | CAACACGGTAACCGACACGCTGCGCGGCAGCGGCGCCTGGGGGCTGCTGCTGC |
| | | ACCGCGTGGGCGACGACGTGCTCACCCACCTGCTGTCGCGCTGCGCGCTCTAC |
| | | CTGCTGGTGCCCCCGACCTGCGCCTACCAGGTGTGTGGGCCGCCGCTCTATGA |
| | | CCTCCGCGCCGCCGCCGCCGCCGCTCGTCGGCCCACGCGGCAAGTGGGCGGGA |
| | | CCCGGGCGGGCTTCGGACTCCCGCGCCCGGCCTCGTCGAACGGCGGCCACGGG |
| | | GAGGCCGAAGGACTCCTGGAGGCGCGGGCCCAGGGCGCGAGGCGGCGTCGCAG |
| | | TAGCGCGCGGGGACGACTGCCTCCAGCCAAGAGGCCCAGGCGCGGCCTGGAGC |
| | | CCGGGCGGGATCTCGAAGGGCAGGTGGCCCGCAGCCCGCCCCGCGTGGTGACA |
| | | CCTACCCGAGACGCTGCGGAAGCCAAGTCTCGGAAGGGCGACGTGCCCGGGCC |
| | | CTGCCGCCTCTTCCCGGGCGGCGAGCGGGGTGTCGGCTCCGCGTCCTGGCGGC |
| | | TGTCACCCTCGGAGGGCGAGCCGGGTGCCGGAGCTTGCGCTGAGACCAAGAGG |
| | | TTCCTTTACTGCTCCGGCGGTGGCGAACAGCTGCGCCGCTCCTTCCTGCTCTG |
| | | CTCCCTGCCTCCCAGCCTGGCCGGGGCGCGGACACTCGTGGAAACCATCTTTC |
| | | TGGACTCGAAGCCCGGGCCGCCAGGGGCTCCCCGCCGGCCGCGCCGCCTGCCC |
| | | GCGCGCTACTGGCAGATGCGGCCCCTGTTCCGGAAACTGCTTGGGAACCACGC |
| | | GCGGAGCCCCTATGGCGCGCTGCTCAGGGCGCACTGCCCGCTGCCGGCCTCTG |
| | | CGCCCCGGGCGGGGCCAGACCATCAGAAGTGCCCTGGTGTTGGGGGCTGCCCC |
| | | TCTGAGAGGCCGGCCGCTGCCCCCGAGGGCGAGGCGAACTCAGGGCGCCTGGT |
| | | CCAGCTGCTCCGCCAGCACAGCAGCCCCTGGCAGGTGTACGGGCTCCTGCGGG |
| | | CCTGTCTTCGCCGCCTGGTGCCCGCCGGCCTCTGGGGCTCCCGGCACAACGAG |
| | | CGGCGCTTCCTGCGGAACGTGAAGAAGCTCCTCTCCCTGGGGAAGCACGGCAG |
| | | GCTCTCGCAGCAGGAGCTCACGTGGAAGATGAAGGTGCAGGACTGCGCCTGGC |
| | | TGCGCGCGAGCCCAGGGGCTCGCTGCGTGCCCGCCGCGGAGCACCGCCAGCGC |
| | | GAGGCCGTCCTGGGTCGCTTCCTGCACTGGCTGATGGGCGCCTACGTGGTGGA |
| | | GCTGCTCAGGAGCTTCTTCTACGTCACAGAGACCACGTTCCAGAAGAACCGGC |
| | | TCTTCTTCTTCCGGAAGCGCATCTGGAGCCAGCTGCAGCGCCTGGGCGTCAGA |
| | | CAACACTTAGACCGTGTGCGGCTTCGAGAACTGTCAGAAGCAGAGGTCAGGCA |
| | | GCACCAGGAGGCCAGGCCGGCTCTGCTGACATCCAGGCTCCGTTTCGTCCCCA |
| | | AGCCCGGCGGGCTGCGGCCCATCGTGAACGTGGGCTGTGTTGAGGGCGCCCCG |
| | | GCACCGCCCAGAGACAAGAAGGTGCAGCATCTCAGCTCACGGGTCAAGACGCT |
| | | GTTCGCGGTGCTGAACTACGAGCGAGCTCGGCGGCCTGGCCTCCTGGGGGCCT |
| | | CGGTGCTGGGCATGGACGACATCCACAGGGCCTG |
| 3 | Bos taurus | MHRTTRIKITELNPHLMCVLCGGYFIDATTIIECLHSFCKTCIVRYLETSKYC |
| | BMI-1 | PICDVQVHKTRPLLNIRSDKTLQDIVYKLVPGLFKNEMKRRRDFYAAHPSADA |
| | Amino acid | ANGSNEDRGEVADEDKRIITDDEIISLSIEFFDQNRLDRKINKDKEKSKEEVN |
| | | DKRYLRCPAAMTVMHLRKFLRSKMDIPNTFQIDVMYEEEPLKDYYTLMDIAYI |
| | | YTWRRNGPLPLKYRVRPTCKRMKISHQRDGLTNTGELESDSGSDKANSPAGGI |
| | | PSTSSCLPSPSTPVQSPHPQFPHISSTMNGTSSSPSGNHQSSFANRPRKSSVN |
| | | GSSATSSG |
| 4 | Bos taurus | ATGCACAGGACTACACGCATTAAAATCACTGAGCTCAATCCCCACTTGATGTG |
| | BMI-1 | CGTCCTCTGCGGAGGTTACTTCATCGACGCCACTACAATCATTGAGTGTCTTC |
| | Nucleic acid | ATAGTTTTTGCAAGACTTGCATTGTGAGATACCTTGAAACGTCAAAGTATTGT |
| | | CCTATTTGTGATGTCCAGGTGCACAAGACCCGACCTTTGCTTAACATCAGGTC |
| | | AGATAAAACCCTTCAAGACATAGTCTATAAGCTGGTGCCGGGCCTCTTCAAGA |
| | | ACGAAATGAAGAGGCGGCGGGACTTCTATGCAGCCCACCCAAGTGCTGATGCT |
| | | GCGAATGGGTCAAATGAAGATAGGGGTGAAGTGGCTGATGAAGACAAAAGAAT |
| | | CATCACGGACGATGAGATAATTAGTCTTTCCATTGAATTCTTCGATCAAAATA |
| | | GATTGGACCGCAAAATCAACAAGGACAAAGAAAAAAGTAAAGAGGAGGTCAAC |
| | | GACAAGCGGTATTTGCGCTGTCCGGCAGCGATGACAGTGATGCACCTCAGGAA |
| | | GTTCCTTAGATCTAAGATGGACATACCGAACACCTTTCAAATTGATGTTATGT |
| | | ATGAAGAGGAACCCCTCAAAGATTACTACACGCTTATGGATATTGCCTACATA |
| | | TATACATGGCGAAGGAACGGTCCACTTCCACTGAAATATAGGGTGCGACCTAC |
| | | GTGCAAGCGCATGAAGATAAGCCACCAGCGGGATGGACTTACAAATACGGGTG |
| | | AACTGGAAAGCGATAGCGGGTCTGATAAAGCCAATAGCCCAGCTGGCGGAATA |
| | | CCATCTACATCCAGTTGTCTGCCCAGTCCCTCCACGCCAGTGCAAAGTCCACA |
| | | TCCACAATTTCCTCATATAAGCTCCACGATGAACGGAACCTCTAGCAGTCCCT |
| | | CTGGAAACCATCAGAGTTCTTTCGCTAACCGACCGCGGAAGTCTTCAGTTAAT |
| | | GGTAGCTCTGCAACAAGCTCCGGCTGA |
| 5 | Bos taurus | MAHQLLCCEMETIRRAYPDANLLNDRVLRAMLKAEETCAPSVSYFKCVQKEIL |
| | CDK4 | PSMRKIVATWMLEVCEEQKCEEEVFPLAMNYLDRFLSLEPVKKSRLQLLGATC |
| | Amino acid | MFVASKMKETIPLTAEKLCIYTDNSIRPDELLHMELVLVNKLKWNLAAMTPHD |
| | | FIEHFLSKMPVAEENKQIIRKHAQTFVALCATDVKFISNPPSMVAAGSVAAAA |
| | | QGLHLGSANGFLSYHRLTRFLSKVIRCDPDCLRACQEQIEALLESSLRQAQQQ |
| | | NLDPKAAEEEEEEEEVDLACTPTDVRDVNI |
| 6 | Bos taurus | ATGGCACATCAGCTGCTGTGTTGTGAAATGGAGACCATTCGGCGCGCCTATCC |
| | CDK4 | CGATGCTAATCTTCTTAATGATCGAGTGCTGCGGGCCATGCTGAAGGCTGAAG |
| | Nucleic acid | AAACGTGCGCTCCGTCAGTCTCTTATTTCAAATGCGTGCAAAAGGAGATTCTC |
| | | CCTAGTATGCGAAAAATTGTGGCCACATGGATGCTGGAGGTGTGTGAAGAGCA |
| | | GAAGTGTGAAGAAGAGGTCTTCCCACTTGCAATGAACTATCTTGATAGATTTC |
| | | TTAGTCTGGAGCCGGTGAAAAAGTCACGGCTCCAGTTGCTCGGAGCTACTTGC |
| | | ATGTTTGTTGCTTCCAAAATGAAAGAAACAATCCCACTCACAGCAGAGAAGTT |
| | | GTGCATCTACACTGATAACTCTATTCGACCTGACGAGCTGCTTCACATGGAGC |
| | | TGGTTCTCGTCAATAAGCTGAAATGGAACCTCGCAGCGATGACACCCCATGAC |
| | | TTCATAGAGCATTTTCTTTCAAAAATGCCTGTCGCCGAGGAGAATAAGCAGAT |
| | | TATACGCAAACACGCCCAAACATTTGTGGCGCTTTGTGCGACCGACGTCAAAT |
| | | TTATCTCTAACCCACCATCAATGGTTGCCGCTGGCAGCGTTGCTGCCGCGGCT |
| | | CAAGGACTGCATCTGGGCTCAGCAAACGGATTTTTGTCCTATCATCGCCTGAC |
| | | AAGATTCCTGTCTAAAGTCATCAGATGCGATCCCGACTGCTTGCGAGCATGTC |
| | | AGGAACAAATAGAGGCCCTGTTGGAGTCAAGCCTTAGACAGGCGCAACAGCAG |
| | | AATCTCGACCCAAAAGCAGCTGAGGAGGAGGAAGAGGAAGAAGAAGTTGACTT |
| | | GGCTTGTACACCCACGGATGTCAGGGACGTTAACATTTGA |
| 7 | Bos taurus | MAHQLLCCEMETIRRAYPDANLLNDRVLRAMLKAEETCAPSVSYFKCVQKEIL |
| | Cyclin D1 | PSMRKIVATWMLEVCEEQKCEEEVFPLAMNYLDRFLSLEPVKKSRLQLLGATC |
| | Amino acid | MFVASKMKETIPLTAEKLCIYTDNSIRPDELLHMELVLVNKLKWNLAAMTPHD |
| | | FIEHFLSKMPVAEENKQIIRKHAQTFVALCATDVKFISNPPSMVAAGSVAAAA |
| | | QGLHLGSANGFLSYHRLTRFLSKVIRCDPDCLRACQEQIEALLESSLRQAQQQ |
| | | NLDPKAAEEEEEEEEVDLACTPTDVRDVNI* |
| 8 | Bos taurus | ATGGCACATCAGCTGCTGTGTTGTGAAATGGAGACCATTCGGCGCGCCTATCC |
| | Cyclin D1 | CGATGCTAATCTTCTTAATGATCGAGTGCTGCGGGCCATGCTGAAGGCTGAAG |
| | Nucleic acid | AAACGTGCGCTCCGTCAGTCTCTTATTTCAAATGCGTGCAAAAGGAGATTCTC |
| | | CCTAGTATGCGAAAAATTGTGGCCACATGGATGCTGGAGGTGTGTGAAGAGCA |
| | | GAAGTGTGAAGAAGAGGTCTTCCCACTTGCAATGAACTATCTTGATAGATTTC |
| | | TTAGTCTGGAGCCGGTGAAAAAGTCACGGCTCCAGTTGCTCGGAGCTACTTGC |
| | | ATGTTTGTTGCTTCCAAAATGAAAGAAACAATCCCACTCACAGCAGAGAAGTT |
| | | GTGCATCTACACTGATAACTCTATTCGACCTGACGAGCTGCTTCACATGGAGC |
| | | TGGTTCTCGTCAATAAGCTGAAATGGAACCTCGCAGCGATGACACCCCATGAC |
| | | TTCATAGAGCATTTTCTTTCAAAAATGCCTGTCGCCGAGGAGAATAAGCAGAT |
| | | TATACGCAAACACGCCCAAACATTTGTGGCGCTTTGTGCGACCGACGTCAAAT |
| | | TTATCTCTAACCCACCATCAATGGTTGCCGCTGGCAGCGTTGCTGCCGCGGCT |
| | | CAAGGACTGCATCTGGGCTCAGCAAACGGATTTTTGTCCTATCATCGCCTGAC |
| | | AAGATTCCTGTCTAAAGTCATCAGATGCGATCCCGACTGCTTGCGAGCATGTC |
| | | AGGAACAAATAGAGGCCCTGTTGGAGTCAAGCCTTAGACAGGCGCAACAGCAG |
| | | AATCTCGACCCAAAAGCAGCTGAGGAGGAGGAAGAGGAAGAAGAAGTTGACTT |
| | | GGCTTGTACACCCACGGATGTCAGGGACGTTAACATTTGA |
| 9 | Bos taurus | MDEGYFCAALVGEDQPLCPDLPELDLSELDVNDLDTDSFLGGLKWCSDQSEII |
| | PGC1alpha | SNQYNNEPSNIFEKIDEENEANLLAVLTETLDSLPVDEDGLPSFDALTDGDVT |
| | Amino acid | TENEASPSSMPDGTPPPQEAEEPSLLKKLLLAPANTQLSYNECSGLSTQNHAN |
| | | HNHRIRTNPAVVKTENSWSNKAKSICQQQKPQRRPCSELLKYLTTNDDPPHTK |
| | | PTENRNSSRDKCTSKKKAHTQSQTQHLQAKPTTLSLPLTPESPNDPKGSPFEN |
| | | KTIERTLSVELSGTAGLTPPTTPPHKANQDNPFRASPKLKPSCKTVVPPPSKK |
| | | ARYSESSCTQGSNSTKKGPEQSELYAQLSKTSVLTSGHEERKAKRPSLRLFGD |
| | | HDYCQSINSKTEILVSTSQELHDSRQLENKDAPSSNGPGQIHSSTDSDPCYLR |
| | | ETAEVSRQVSPGSTRKQLQDQEIRAELNKHFGHPSQAVFDDKADKTSELRDSD |
| | | FSNEQFSKLPMFINSGLAMDGLFDDSEDESDKLNSPWDGTQSYSLFDVSPSCS |
| | | SFNSPCRDSVSPPKSLFSQRPQRMRSRSRSFSRHRSCSRSPYSRSRSRSPGSR |
| | | SSSRSCYYYESGHCRHRTHRNSPLCASRSRSPHSRRPRYDSYEEYQHERLKRE |
| | | EYRREYEKRESERAKQRERQRQKAINQQVSSHFPLQEERRVIYVG |
| 10 | Bos taurus | ATGGATGAGGGGTACTTTTGTGCTGCACTCGTCGGCGAGGACCAACCCCTTTG |
| | PGC1alpha | CCCGGACCTCCCCGAGTTGGACCTCAGTGAGCTTGACGTCAACGATTTGGACA |
| | Nucleic acid | CGGATAGCTTTCTGGGTGGGCTTAAATGGTGTTCTGACCAATCCGAGATCATA |
| | | TCCAATCAGTATAATAACGAACCGAGTAACATTTTCGAGAAAATAGATGAGGA |
| | | GAATGAAGCAAACCTTCTGGCTGTGCTCACGGAGACCTTGGATTCCCTCCCTG |
| | | TGGACGAGGACGGTCTTCCTAGCTTTGACGCGCTCACAGATGGAGATGTTACA |
| | | ACAGAGAATGAAGCCTCCCCTTCAAGTATGCCAGATGGAACTCCACCCCCGCA |
| | | AGAAGCTGAGGAACCTTCCCTGCTTAAGAAACTGCTGTTGGCTCCGGCAAACA |
| | | CACAACTCTCATATAATGAATGCTCTGGGCTGTCAACCCAAAACCATGCGAAT |
| | | CATAACCACAGAATTAGGACAAATCCAGCGGTCGTGAAGACGGAAAATTCCTG |
| | | GTCTAATAAGGCAAAGTCTATATGTCAACAACAGAAGCCGCAGAGAAGGCCCT |
| | | GTTCTGAACTGCTTAAATATCTTACCACGAACGACGACCCCCCACACACGAAG |
| | | CCCACTGAAAACCGAAATTCTTCAAGGGATAAATGTACCTCTAAAAAAAAGGC |
| | | CCATACACAATCCCAGACTCAACATTTGCAAGCTAAACCGACTACGCTTTCTC |
| | | TTCCCCTCACTCCGGAAAGCCCCAACGACCCCAAAGGTTCCCCATTTGAGAAT |
| | | AAAACGATAGAACGCACCCTGTCCGTCGAGTTGTCAGGTACGGCCGGCCTTAC |
| | | TCCGCCTACTACTCCACCTCATAAGGCGAACCAAGATAATCCCTTCAGAGCTT |
| | | CACCAAAGCTTAAGCCAAGTTGCAAAACTGTCGTCCCCCCTCCGTCTAAGAAA |
| | | GCCCGCTATTCCGAAAGTTCATGCACCCAAGGCTCTAACTCTACCAAAAAGGG |
| | | GCCTGAACAGTCAGAATTGTATGCACAGTTGAGTAAAACGAGCGTTCTCACCT |
| | | CTGGACATGAGGAACGAAAGGCTAAGCGGCCCTCCCTTAGGTTGTTCGGGGAC |
| | | CATGACTACTGCCAGTCCATAAATTCCAAAACAGAAATACTTGTGTCCACTTC |
| | | ACAAGAGCTTCATGACTCTAGACAACTTGAAAACAAGGATGCTCCTTCAAGCA |
| | | ATGGTCCCGGGCAGATTCACAGTTCTACCGACAGCGACCCCTGTTATCTTAGA |
| | | GAGACAGCGGAGGTTAGTAGGCAGGTGTCCCCCGGATCAACACGAAAACAGCT |
| | | CCAAGACCAGGAAATACGAGCGGAGCTGAATAAACACTTTGGCCACCCGAGCC |
| | | AAGCTGTCTTCGACGACAAAGCCGATAAAACTAGCGAGCTGAGGGATAGCGAC |
| | | TTCTCTAATGAACAATTCTCTAAACTTCCAATGTTCATAAATAGCGGCCTCGC |
| | | TATGGACGGCCTCTTCGATGATTCCGAGGACGAATCTGACAAGTTGAATAGTC |
| | | CCTGGGACGGGACCCAAAGTTACTCCCTGTTCGATGTGTCACCCTCATGTTCC |
| | | TCATTCAACTCCCCGTGTAGAGACTCCGTCTCCCCTCCAAAAAGCCTTTTTAG |
| | | TCAGCGGCCTCAAAGGATGCGATCTCGCAGTCGATCCTTTAGTAGGCACAGAA |
| | | GTTGTTCAAGATCTCCATATTCTCGGTCACGGTCTAGAAGTCCTGGTTCCAGA |
| | | AGTTCATCCAGATCATGTTACTACTACGAAAGCGGACATTGTAGGCACAGGAC |
| | | CCACCGGAATTCTCCACTGTGCGCTAGTCGAAGTCGATCTCCTCATAGCAGAC |
| | | GGCCACGATATGACTCCTACGAAGAGTACCAGCACGAGCGGTTGAAGCGGGAA |
| | | GAGTACCGGCGCGAGTACGAAAAAAGGGAAAGCGAAAGAGCCAAACAGCGCGA |
| | | GAGGCAGAGACAAAAAGCGATTAATCAACAGGTCAGTAGCCATTTCCCTCTGC |
| | | AAGAGGAACGCAGGGTGATTTATGTCGGA |
| 11 | Bos taurus | MSVGPACPQSLLGPEASNSRESSPMPEESYVSLQTSSADTLDTDTVSPLPSSM |
| | Nanog | DLLIQDSPDSSTSPRVKPLSPSVEESTEKEETVPVKKQKIRTVFSQTQLCVLN |
| | Amino acid | DRFQRQKYLSLQQMQELSNILNLSYKQVKTWFQNQRMKCKKWQKNNWPRNSNG |
| | | MPQGPAMAEYPGFYSYHQGCLVNSPGNLPMWGNQTWNNPTWSNQSWNSQSWSN |
| | | HSWNSQAWCPQAWNNQPWNNQFNNYMEEFLQPGIQLQQNSPVCDLEATLGTAG |
| | | ENYNVIQQTVKYFNSQQQITDLFPNYPLNIQPEDL |
| 12 | Bos taurus | ATGAGCGTGGGGCCGGCTTGCCCTCAATCTCTGCTTGGCCCGGAGGCCTCTAA |
| | Nanog | TAGCCGTGAGAGCAGTCCTATGCCTGAGGAAAGTTATGTATCCCTGCAAACCT |
| | Nucleic acid | CCAGTGCCGATACGTTGGACACCGATACCGTGTCACCCCTGCCATCTTCAATG |
| | | GACCTTCTGATTCAAGACTCACCCGATAGCTCCACCAGTCCCCGCGTGAAGCC |
| | | CCTGTCCCCAAGCGTTGAAGAGAGCACCGAGAAGGAAGAGACAGTCCCTGTGA |
| | | AAAAGCAGAAGATCCGGACTGTTTTCAGCCAGACGCAATTGTGCGTGTTGAAC |
| | | GACCGCTTCCAGCGTCAGAAGTACCTGAGCCTGCAACAGATGCAGGAGCTGAG |
| | | TAACATCCTCAACCTGTCCTACAAACAGGTCAAGACCTGGTTCCAGAATCAAC |
| | | GGATGAAGTGTAAGAAATGGCAAAAGAATAACTGGCCCCGTAACTCCAATGGG |
| | | ATGCCCCAAGGTCCCGCTATGGCAGAATACCCAGGGTTCTACTCCTACCACCA |
| | | GGGCTGCCTGGTAAACAGCCCTGGCAACCTCCCTATGTGGGGCAACCAGACTT |
| | | GGAATAACCCTACATGGTCTAATCAGAGTTGGAACTCTCAGAGCTGGTCTAAT |
| | | CACTCTTGGAACTCACAAGCCTGGTGCCCTCAAGCTTGGAATAACCAGCCATG |
| | | GAATAACCAGTTCAATAACTACATGGAAGAGTTCCTGCAGCCAGGTATTCAGC |
| | | TGCAACAGAACAGCCCCGTGTGTGACCTGGAGGCCACCCTGGGAACCGCCGGC |
| | | GAGAACTATAATGTGATCCAACAGACCGTCAAGTATTTCAATTCTCAGCAACA |
| | | GATCACGGACCTGTTCCCGAACTACCCTCTGAACATCCAGCCTGAGGACTTGT |
| | | AA |
| 13 | Bos taurus | MADAEVLILPKKHKKKKERKSLPEEAVAEIQHAEEFLIKPESRVAQLDTSQWP |
| | DKC1 | LLLKNFDKLNVRTTHYTPLPCGSNPLKREIGDYIRTGFINLDKPSNPSSHEVV |
| | Amino acid | AWIRRILRVEKTGHSGTLDPKVTGCLIVCIERATRLVKSQQSAGKEYVGIVRL |
| | | HNAIEGGTQLSRALETLTGALFQRPPLIAAVKRQLRVRTIYESKMIEYDPERR |
| | | LGIFWVSCEAGTYIRTLCVHLGLLLGVGGQMQELRRVRSGVMSEKDHMVTMHD |
| | | VLDAQWLYDNHKDESYLRRVVYPLEKLLTSHKRLVMKDSAVNAICYGAKIMLP |
| | | GVLRYEDGIEVNQEIVVITTKGEAICMAIALMTTAVISTCDHGIVAKIKRVIM |
| | | ERDTYPRKWGLGPKASQKKLMIKQGLLDKHGKPTDSTPATWMQEYVDYSNSAK |
| | | KDVPPKAVKATPVVAEVVKTPKRKRESESEDTSPAAPQVVKKEKKKKKKEKAK |
| | | AAAEKPGAGDSDSTKKKKKKKIKAEMVSE |
| 14 | Bos taurus | ATGGCAGATGCTGAAGTTCTTATTCTCCCTAAGAAACACAAAAAGAAAAAAGA |
| | DKC1 | ACGCAAATCACTCCCAGAGGAGGCCGTTGCAGAGATACAACATGCGGAAGAGT |
| | Nucleic acid | TCCTGATCAAACCAGAATCTAGAGTTGCTCAACTGGATACATCTCAATGGCCT |
| | | TTGCTGCTGAAAAATTTCGATAAACTCAACGTCAGAACAACCCACTATACCCC |
| | | GCTCCCGTGTGGGTCTAATCCGCTTAAACGAGAAATCGGTGACTACATTCGGA |
| | | CAGGTTTTATTAATCTCGATAAGCCTAGCAACCCCTCTAGTCACGAGGTTGTC |
| | | GCTTGGATCCGAAGGATATTGAGGGTCGAGAAGACAGGGCACTCAGGTACCCT |
| | | TGATCCCAAAGTCACCGGCTGCCTCATAGTTTGCATCGAAAGAGCCACTCGAC |
| | | TGGTTAAATCCCAACAATCTGCAGGAAAAGAGTATGTTGGCATAGTCAGGCTT |
| | | CATAATGCGATTGAGGGTGGCACCCAACTCTCCAGGGCACTCGAAACACTTAC |
| | | GGGGGCACTTTTCCAGCGACCACCTCTTATAGCGGCAGTCAAACGGCAACTCC |
| | | GAGTTAGGACGATATACGAGAGCAAGATGATCGAGTATGATCCTGAGAGACGG |
| | | CTTGGTATCTTTTGGGTTAGTTGTGAGGCCGGCACGTATATCCGAACGCTTTG |
| | | TGTGCACTTGGGGTTGCTGCTTGGGGTCGGGGGCCAAATGCAGGAATTGCGGC |
| | | GGGTGCGGTCAGGAGTGATGTCAGAGAAGGACCATATGGTGACGATGCATGAC |
| | | GTCCTCGATGCCCAGTGGCTTTACGACAATCACAAAGATGAGTCATACCTTAG |
| | | GCGAGTTGTCTACCCACTCGAAAAGTTGCTCACTTCCCACAAACGACTTGTGA |
| | | TGAAAGATAGCGCCGTGAACGCGATCTGCTATGGCGCTAAGATAATGCTCCCC |
| | | GGTGTCCTCAGATACGAAGACGGGATTGAAGTTAATCAAGAAATCGTTGTCAT |
| | | TACGACTAAAGGAGAGGCCATTTGCATGGCGATCGCCCTGATGACGACTGCCG |
| | | TCATAAGTACATGCGATCATGGAATAGTGGCAAAAATCAAGAGGGTCATCATG |
| | | GAAAGGGACACATACCCCCGGAAATGGGGTCTGGGACCGAAAGCCTCTCAAAA |
| | | GAAACTGATGATTAAACAGGGACTTCTTGACAAACATGGTAAACCGACTGATA |
| | | GCACTCCCGCTACATGGATGCAAGAATATGTGGACTATTCTAATTCCGCTAAA |
| | | AAGGATGTGCCGCCTAAAGCAGTGAAAGCGACCCCCGTCGTTGCTGAAGTGGT |
| | | TAAAACGCCAAAACGCAAAAGAGAGTCTGAATCAGAAGACACTTCTCCCGCAG |
| | | CGCCGCAGGTCGTCAAAAAGGAGAAGAAAAAAAAGAAGAAAGAGAAGGCGAAA |
| | | GCTGCCGCTGAGAAGCCGGGTGCCGGTGACAGCGATAGTACAAAGAAGAAAAA |
| | | AAAAAAGAAAATCAAGGCTGAAATGGTTTCTGAGTAG |
| 15 | Bos taurus | MDPGPPPPQPAPQGQGPPPAQAPQGQGPPSAPGQPAPPGPPAAPQAPPAGHQI |
| | YAP | VHVRGDSETDLEALFNAVMNPKTANVPQTVPMRLRKLPDSFFKPPEPKSHSRQ |
| | Amino acid | ASTDAGTAGALTPQHVRAHSSPASLQLGAVSPGTLTPTGVVSGPAAAPAAQHL |
| | | RQSSFEIPDDVPLPAGWEMAKTSSGQRYFLNHIDQTTTWQDPRKAMLSQMNVT |
| | | APTSPPVQQNMMNSASGPLPDGWEQAMTQDGEIYYINHKNKTTSWLDPRLDPR |
| | | FAMNQRISQSAPVKQPPPLAPQSPQGGVLGGGSSNQQQQMRLQQLQMEKERLR |
| | | LKQQELLRQVRPQAMRNINPSTANSPKCQELALRSQLPTLEQDGGTQNPVSSP |
| | | GMSQELRTMTTNSSDPFLNSGTYHSRDESTDSGLSMSSYSVPRTPDDFLNSVD |
| | | EMDTGDTINQSTLPSQQNRFPDYLEAIPGTNVDLGTLEGDGMNIEGEELMPSL |
| | | QEALSSDILNDMESVLAATKLDKESFLTWL |
| 16 | Bos taurus | ATGGATCCTGGACCGCCTCCCCCGCAGCCTGCGCCCCAGGGACAGGGGCCACC |
| | YAP | CCCAGCGCAGGCCCCACAAGGCCAGGGTCCCCCAAGCGCTCCAGGCCAGCCTG |
| | Nucleic acid | CACCACCCGGCCCTCCCGCTGCACCCCAGGCCCCACCTGCCGGTCACCAGATC |
| | | GTCCACGTCCGCGGGGACTCCGAGACAGATCTGGAAGCTCTCTTTAACGCGGT |
| | | GATGAACCCAAAGACGGCAAATGTGCCTCAGACCGTCCCTATGCGCCTGAGGA |
| | | AGCTGCCCGACTCCTTTTTCAAACCTCCGGAGCCAAAATCCCATTCTCGCCAG |
| | | GCCTCCACGGATGCCGGCACCGCGGGCGCACTGACGCCCCAGCACGTCAGGGC |
| | | TCATTCTAGCCCTGCCAGCCTTCAGCTCGGCGCTGTGAGCCCCGGGACCCTGA |
| | | CCCCTACGGGAGTTGTGTCCGGCCCCGCCGCAGCCCCTGCCGCACAGCACCTG |
| | | CGCCAATCCTCTTTTGAGATTCCCGATGACGTGCCTCTGCCCGCCGGTTGGGA |
| | | GATGGCTAAGACCTCTAGCGGCCAGAGGTACTTCCTCAATCACATTGACCAGA |
| | | CTACCACTTGGCAGGACCCCCGGAAGGCGATGCTTTCTCAGATGAACGTGACA |
| | | GCGCCGACTTCCCCTCCCGTCCAACAGAACATGATGAACTCCGCCAGCGGCCC |
| | | ACTCCCCGATGGGTGGGAGCAGGCTATGACCCAGGACGGTGAGATTTATTACA |
| | | TCAACCACAAGAACAAGACGACCTCTTGGCTGGATCCAAGATTGGATCCGCGC |
| | | TTCGCCATGAACCAGAGAATCTCACAGTCCGCCCCTGTGAAGCAGCCGCCCCC |
| | | GTTGGCGCCGCAAAGCCCTCAGGGCGGAGTGCTGGGCGGTGGATCTTCCAACC |
| | | AGCAACAGCAAATGCGCTTGCAGCAATTGCAGATGGAAAAAGAGAGGCTGCGC |
| | | CTCAAACAACAGGAACTTCTGAGGCAGGTGCGCCCCCAGGCCATGAGGAACAT |
| | | CAACCCGTCCACCGCTAATTCTCCTAAGTGCCAGGAACTGGCTCTGCGCTCTC |
| | | AGCTGCCCACGCTGGAGCAGGACGGCGGGACCCAGAATCCTGTTAGTTCTCCG |
| | | GGAATGAGCCAAGAACTGCGTACTATGACTACAAACAGCTCCGACCCATTCTT |
| | | GAACAGCGGCACCTACCACTCTCGTGATGAGAGCACCGACTCAGGATTGTCCA |
| | | TGAGCTCCTATAGCGTGCCTCGTACGCCCGATGACTTCCTCAACAGCGTGGAC |
| | | GAAATGGATACCGGTGACACCATCAACCAAAGCACCCTCCCAAGCCAGCAAAA |
| | | CCGTTTCCCAGACTACTTGGAGGCAATCCCGGGGACAAACGTGGATCTTGGAA |
| | | CTCTGGAGGGCGACGGGATGAACATCGAGGGCGAAGAGCTCATGCCTTCCCTC |
| | | CAGGAGGCGCTGAGCTCCGACATCCTGAACGATATGGAATCCGTGTTGGCTGC |
| | | AACCAAGCTGGACAAGGAGTCCTTCTTGACATGGTTGTGA |
| 17 | 5′ ITR | TTAACCCTAGAAAGATAGTCTGCGTAAAATTGACGCATGCATTCTTGAAATAT |
| | | TGCTCTCTCTTTCTAAATAGCGCGAATCCGTCGCTGTGCATTTAGGACATCTC |
| | | AGTCGCCGCTTGGAGCTCCCGTGAGGCGTGCTTGTCAATGCGGTAAGTGTCAC |
| | | TGATTTTGAACTATAACGACCGCGTGAGTCAAAATGACGCATGATTATCTTTT |
| | | ACGTGACTTTTAAGATTTAACTCATACGATAATTATATTGTTATTTCATGTTC |
| | | TACTTACGTGATAACTTATTATATATATATTTTCTTGTTATAGATATC |
| 18 | 3′ ITR | TTAACCCTAGAAAGATAATCATATTGTGACGTACGTTAAAGATAATCATGCGT |
| | | AAAATTGACGCATGTGTTTTATCGGTCTGTATATCGAGGTTTATTTATTAATT |
| | | TGAATAGATATTAAGTTTTATTATATTTACACTTACATACTAATAATAAATTC |
| | | AACAAA |

What is claimed is:

1. A method for generating a cell line suitable for dietary consumption, the method comprising:
(a) introducing into a population of cells an exogenous nucleic acid sequence comprising a first polynucleotide encoding a gene of interest located between a 5′ inverted terminal repeat (ITR) and a 3′ inverted terminal repeat (ITR), and a second polynucleotide encoding a selection marker located outside of the 5′ ITR and the 3′ ITR,
(b) selecting a transduced cell from the population of cells using the selection marker; and
(c) culturing the transduced cells under conditions sufficient to expand the transduced cells.

2. The method of claim 1, further comprising a second selecting step.

3. The method of claim 2, wherein the second selecting step comprises using the second selection marker to further select the transduced cell.

4. The method of claim 2, wherein the second selecting step is performed 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the first selecting step.

5. The method of claim 1, wherein the episomal polynucleotide encoding the selection marker remains in the cell following introduction for at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, or at most 30 days.

6. The method of claim 1, wherein the cell line is derived from: livestock, poultry, or game animal species.

7. The method of claim 1, wherein the cell line comprises cells selected from: fibroblasts, mesenchymal cells, chondrocytes, myoblasts, myocytes, satellite cells, side population cells, muscle derived stem cells, myofibroblasts, mesenchymal stem cells, myogenic pericytes, or mesoangioblasts.

8. The method of claim 1, wherein the 5′ ITR and the 3′ ITR are transposon ITRs, and wherein the first polynucleotide is integrated into the genome while the second polynucleotide episomal.


SEQUENCE LISTING (18 sequences)

SEQ ID NO: 1 - protein, 682 aa, Bos taurus
MPRAPRCRAVRALLRASYRQVLPLAAFVRRLRPQGHRLVRRGDPAAFRALVAQCLVCVPWDAQPPPAAPSFRQVSCLKELVARVVQRLCERGARNVLAFGFTLLAGARGGPPVAFTTSVRSYLPNTVTDTLRGSGAWGLLLHRVGDDVLTHLLSRCALYLLVPPTCAYQVCGPPLYDLRAAAAAARRPTRQVGGTRAGFGLPRPASSNGGHGEAEGLLEARAQGARRRRSSARGRLPPAKRPRRGLEPGRDLEGQVARSPPRVVTPTRDAAEAKSRKGDVPGPCRLFPGGERGVGSASWRLSPSEGEPGAGACAETKRFLYCSGGGEQLRRSFLLCSLPPSLAGARTLVETIFLDSKPGPPGAPRRPRRLPARYWQMRPLFRKLLGNHARSPYGALLRAHCPLPASAPRAGPDHQKCPGVGGCPSERPAAAPEGEANSGRLVQLLRQHSSPWQVYGLLRACLRRLVPAGLWGSRHNERRFLRNVKKLLSLGKHGRLSQQELTWKMKVQDCAWLRASPGARCVPAAEHRQREAVLGRFLHWLMGAYVVELLRSFFYVTETTFQKNRLFFFRKRIWSQLQRLGVRQHLDRVRLRELSEAEVRQHQEARPALLTSRLRFVPKPGGLRPIVNVGCVEGAPAPPRDKKVQHLSSRVKTLFAVLNYERARRPGLLGASVLGMDDIHRA

SEQ ID NO: 2 - DNA, 2048 bp, Bos taurus
  source 1..2048: mol_type = genomic DNA
atgccgcgcgcgcccaggtgccgggccgtgcgcgcccttctgcgggccagctaccggcaggtgctgcccctggccgccttcgtacggcgcctgcggccccagggccaccggcttgtgcggcgcggggacccggcggccttccgcgcgctggtggctcagtgcttggtgtgcgtgccctgggacgcgcagccgccccctgccgccccgtccttccgccaggtgtcctgcctgaaggagctggtggccagagtcgtgcagaggctctgcgagcgcggcgcgaggaacgtgctggccttcggcttcacgctgctggccggggcccgcggcgggccgcccgtggccttcacgaccagcgtacgcagctacctgcccaacacggtaaccgacacgctgcgcggcagcggcgcctgggggctgctgctgcaccgcgtgggcgacgacgtgctcacccacctgctgtcgcgctgcgcgctctacctgctggtgcccccgacctgcgcctaccaggtgtgtgggccgccgctctatgacctccgcgccgccgccgccgccgctcgtcggcccacgcggcaagtgggcgggacccgggcgggcttcggactcccgcgcccggcctcgtcgaacggcggccacggggaggccgaaggactcctggaggcgcgggcccagggcgcgaggcggcgtcgcagtagcgcgcggggacgactgcctccagccaagaggcccaggcgcggcctggagcccgggcgggatctcgaagggcaggtggcccgcagcccgccccgcgtggtgacacctacccgagacgctgcggaagccaagtctcggaagggcgacgtgcccgggccctgccgcctcttcccgggcggcgagcggggtgtcggctccgcgtcctggcggctgtcaccctcggagggcgagccgggtgccggagcttgcgctgagaccaagaggttcctttactgctccggcggtggcgaacagctgcgccgctccttcctgctctgctccctgcctcccagcctggccggggcgcggacactcgtggaaaccatctttctggactcgaagcccgggccgccaggggctccccgccggccgcgccgcctgcccgcgcgctactggcagatgcggcccctgttccggaaactgcttgggaaccacgcgcggagcccctatggcgcgctgctcagggcgcactgcccgctgccggcctctgcgccccgggcggggccagaccatcagaagtgccctggtgttgggggctgcccctctgagaggccggccgctgcccccgagggcgaggcgaactcagggcgcctggtccagctgctccgccagcacagcagcccctggcaggtgtacgggctcctgcgggcctgtcttcgccgcctggtgcccgccggcctctggggctcccggcacaacgagcggcgcttcctgcggaacgtgaagaagctcctctccctggggaagcacggcaggctctcgcagcaggagctcacgtggaagatgaaggtgcaggactgcgcctggctgcgcgcgagcccaggggctcgctgcgtgcccgccgcggagcaccgccagcgcgaggccgtcctgggtcgcttcctgcactggctgatgggcgcctacgtggtggagctgctcaggagcttcttctacgtcacagagaccacgttccagaagaaccggctcttcttcttccggaagcgcatctggagccagctgcagcgcctgggcgtcagacaacacttagaccgtgtgcggcttcgagaactgtcagaagcagaggtcaggcagcaccaggaggccaggccggctctgctgacatccaggctccgtttcgtccccaagcccggcgggctgcggcccatcgtgaacgtgggctgtgttgagggcgccccggcaccgcccagagacaagaaggtgcagcatctcagctcacgggtcaagacgctgttcgcggtgctgaactacgagcgagctcggcggcctggcctcctgggggcctcggtgctgggcatggacgacatccacagggcctg

SEQ ID NO: 3 - protein, 326 aa, Bos taurus
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEDKRIITDDEIISLSIEFFDQNRLDRKINKDKEKSKEEVNDKRYLRCPAAMTVMHLRKFLRSKMDIPNTFQIDVMYEEEPLKDYYTLMDIAYIYTWRRNGPLPLKYRVRPTCKRMKISHQRDGLTNTGELESDSGSDKANSPAGGIPSTSSCLPSPSTPVQSPHPQFPHISSTMNGTSSSPSGNHQSSFANRPRKSSVNGSSATSSG

SEQ ID NO: 4 - DNA, 981 bp, Bos taurus
  source 1..981: mol_type = genomic DNA
atgcacaggactacacgcattaaaatcactgagctcaatccccacttgatgtgcgtcctctgcggaggttacttcatcgacgccactacaatcattgagtgtcttcatagtttttgcaagacttgcattgtgagataccttgaaacgtcaaagtattgtcctatttgtgatgtccaggtgcacaagacccgacctttgcttaacatcaggtcagataaaacccttcaagacatagtctataagctggtgccgggcctcttcaagaacgaaatgaagaggcggcgggacttctatgcagcccacccaagtgctgatgctgcgaatgggtcaaatgaagataggggtgaagtggctgatgaagacaaaagaatcatcacggacgatgagataattagtctttccattgaattcttcgatcaaaatagattggaccgcaaaatcaacaaggacaaagaaaaaagtaaagaggaggtcaacgacaagcggtatttgcgctgtccggcagcgatgacagtgatgcacctcaggaagttccttagatctaagatggacataccgaacacctttcaaattgatgttatgtatgaagaggaacccctcaaagattactacacgcttatggatattgcctacatatatacatggcgaaggaacggtccacttccactgaaatatagggtgcgacctacgtgcaagcgcatgaagataagccaccagcgggatggacttacaaatacgggtgaactggaaagcgatagcgggtctgataaagccaatagcccagctggcggaataccatctacatccagttgtctgcccagtccctccacgccagtgcaaagtccacatccacaatttcctcatataagctccacgatgaacggaacctctagcagtccctctggaaaccatcagagttctttcgctaaccgaccgcggaagtcttcagttaatggtagctctgcaacaagctccggctga

SEQ ID NO: 5 - protein, 295 aa, Bos taurus
MAHQLLCCEMETIRRAYPDANLLNDRVLRAMLKAEETCAPSVSYFKCVQKEILPSMRKIVATWMLEVCEEQKCEEEVFPLAMNYLDRFLSLEPVKKSRLQLLGATCMFVASKMKETIPLTAEKLCIYTDNSIRPDELLHMELVLVNKLKWNLAAMTPHDFIEHFLSKMPVAEENKQIIRKHAQTFVALCATDVKFISNPPSMVAAGSVAAAAQGLHLGSANGFLSYHRLTRFLSKVIRCDPDCLRACQEQIEALLESSLRQAQQQNLDPKAAEEEEEEEEVDLACTPTDVRDVNI

SEQ ID NO: 6 - DNA, 888 bp, Bos taurus
  source 1..888: mol_type = genomic DNA
atggcacatcagctgctgtgttgtgaaatggagaccattcggcgcgcctatcccgatgctaatcttcttaatgatcgagtgctgcgggccatgctgaaggctgaagaaacgtgcgctccgtcagtctcttatttcaaatgcgtgcaaaaggagattctccctagtatgcgaaaaattgtggccacatggatgctggaggtgtgtgaagagcagaagtgtgaagaagaggtcttcccacttgcaatgaactatcttgatagatttcttagtctggagccggtgaaaaagtcacggctccagttgctcggagctacttgcatgtttgttgcttccaaaatgaaagaaacaatcccactcacagcagagaagttgtgcatctacactgataactctattcgacctgacgagctgcttcacatggagctggttctcgtcaataagctgaaatggaacctcgcagcgatgacaccccatgacttcatagagcattttctttcaaaaatgcctgtcgccgaggagaataagcagattatacgcaaacacgcccaaacatttgtggcgctttgtgcgaccgacgtcaaatttatctctaacccaccatcaatggttgccgctggcagcgttgctgccgcggctcaaggactgcatctgggctcagcaaacggatttttgtcctatcatcgcctgacaagattcctgtctaaagtcatcagatgcgatcccgactgcttgcgagcatgtcaggaacaaatagaggccctgttggagtcaagccttagacaggcgcaacagcagaatctcgacccaaaagcagctgaggaggaggaagaggaagaagaagttgacttggcttgtacacccacggatgtcagggacgttaacatttga

SEQ ID NO: 7 - protein, 295 aa, Bos taurus; identical to SEQ ID NO: 5

SEQ ID NO: 8 - DNA, 888 bp, Bos taurus; identical to SEQ ID NO: 6
  source 1..888: mol_type = genomic DNA

SEQ ID NO: 9 - protein, 681 aa, Bos taurus
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTENEASPSSMPDGTPPPQEAEEPSLLKKLLLAPANTQLSYNECSGLSTQNHANHNHRIRTNPAVVKTENSWSNKAKSICQQQKPQRRPCSELLKYLTTNDDPPHTKPTENRNSSRDKCTSKKKAHTQSQTQHLQAKPTTLSLPLTPESPNDPKGSPFENKTIERTLSVELSGTAGLTPPTTPPHKANQDNPFRASPKLKPSCKTVVPPPSKKARYSESSCTQGSNSTKKGPEQSELYAQLSKTSVLTSGHEERKAKRPSLRLFGDHDYCQSINSKTEILVSTSQELHDSRQLENKDAPSSNGPGQIHSSTDSDPCYLRETAEVSRQVSPGSTRKQLQDQEIRAELNKHFGHPSQAVFDDKADKTSELRDSDFSNEQFSKLPMFINSGLAMDGLFDDSEDESDKLNSPWDGTQSYSLFDVSPSCSSFNSPCRDSVSPPKSLFSQRPQRMRSRSRSFSRHRSCSRSPYSRSRSRSPGSRSSSRSCYYYESGHCRHRTHRNSPLCASRSRSPHSRRPRYDSYEEYQHERLKREEYRREYEKRESERAKQRERQRQKAINQQVSSHFPLQEERRVIYVG

SEQ ID NO: 10 - DNA, 2043 bp, Bos taurus
  source 1..2043: mol_type = genomic DNA
atggatgaggggtacttttgtgctgcactcgtcggcgaggaccaacccctttgcccggacctccccgagttggacctcagtgagcttgacgtcaacgatttggacacggatagctttctgggtgggcttaaatggtgttctgaccaatccgagatcatatccaatcagtataataacgaaccgagtaacattttcgagaaaatagatgaggagaatgaagcaaaccttctggctgtgctcacggagaccttggattccctccctgtggacgaggacggtcttcctagctttgacgcgctcacagatggagatgttacaacagagaatgaagcctccccttcaagtatgccagatggaactccacccccgcaagaagctgaggaaccttccctgcttaagaaactgctgttggctccggcaaacacacaactctcatataatgaatgctctgggctgtcaacccaaaaccatgcgaatcataaccacagaattaggacaaatccagcggtcgtgaagacggaaaattcctggtctaataaggcaaagtctatatgtcaacaacagaagccgcagagaaggccctgttctgaactgcttaaatatcttaccacgaacgacgaccccccacacacgaagcccactgaaaaccgaaattcttcaagggataaatgtacctctaaaaaaaaggcccatacacaatcccagactcaacatttgcaagctaaaccgactacgctttctcttcccctcactccggaaagccccaacgaccccaaaggttccccatttgagaataaaacgatagaacgcaccctgtccgtcgagttgtcaggtacggccggccttactccgcctactactccacctcataaggcgaaccaagataatcccttcagagcttcaccaaagcttaagccaagttgcaaaactgtcgtcccccctccgtctaagaaagcccgctattccgaaagttcatgcacccaaggctctaactctaccaaaaaggggcctgaacagtcagaattgtatgcacagttgagtaaaacgagcgttctcacctctggacatgaggaacgaaaggctaagcggccctcccttaggttgttcggggaccatgactactgccagtccataaattccaaaacagaaatacttgtgtccacttcacaagagcttcatgactctagacaacttgaaaacaaggatgctccttcaagcaatggtcccgggcagattcacagttctaccgacagcgacccctgttatcttagagagacagcggaggttagtaggcaggtgtcccccggatcaacacgaaaacagctccaagaccaggaaatacgagcggagctgaataaacactttggccacccgagccaagctgtcttcgacgacaaagccgataaaactagcgagctgagggatagcgacttctctaatgaacaattctctaaacttccaatgttcataaatagcggcctcgctatggacggcctcttcgatgattccgaggacgaatctgacaagttgaatagtccctgggacgggacccaaagttactccctgttcgatgtgtcaccctcatgttcctcattcaactccccgtgtagagactccgtctcccctccaaaaagcctttttagtcagcggcctcaaaggatgcgatctcgcagtcgatcctttagtaggcacagaagttgttcaagatctccatattctcggtcacggtctagaagtcctggttccagaagttcatccagatcatgttactactacgaaagcggacattgtaggcacaggacccaccggaattctccactgtgcgctagtcgaagtcgatctcctcatagcagacggccacgatatgactcctacgaagagtaccagcacgagcggttgaagcgggaagagtaccggcgcgagtacgaaaaaagggaaagcgaaagagccaaacagcgcgagaggcagagacaaaaagcgattaatcaacaggtcagtagccatttccctctgcaagaggaacgcagggtgatttatgtcgga

SEQ ID NO: 11 - protein, 300 aa, Bos taurus
MSVGPACPQSLLGPEASNSRESSPMPEESYVSLQTSSADTLDTDTVSPLPSSMDLLIQDSPDSSTSPRVKPLSPSVEESTEKEETVPVKKQKIRTVFSQTQLCVLNDRFQRQKYLSLQQMQELSNILNLSYKQVKTWFQNQRMKCKKWQKNNWPRNSNGMPQGPAMAEYPGFYSYHQGCLVNSPGNLPMWGNQTWNNPTWSNQSWNSQSWSNHSWNSQAWCPQAWNNQPWNNQFNNYMEEFLQPGIQLQQNSPVCDLEATLGTAGENYNVIQQTVKYFNSQQQITDLFPNYPLNIQPEDL

SEQ ID NO: 12 - DNA, 903 bp, Bos taurus
  source 1..903: mol_type = genomic DNA
atgagcgtggggccggcttgccctcaatctctgcttggcccggaggcctctaatagccgtgagagcagtcctatgcctgaggaaagttatgtatccctgcaaacctccagtgccgatacgttggacaccgataccgtgtcacccctgccatcttcaatggaccttctgattcaagactcacccgatagctccaccagtccccgcgtgaagcccctgtccccaagcgttgaagagagcaccgagaaggaagagacagtccctgtgaaaaagcagaagatccggactgttttcagccagacgcaattgtgcgtgttgaacgaccgcttccagcgtcagaagtacctgagcctgcaacagatgcaggagctgagtaacatcctcaacctgtcctacaaacaggtcaagacctggttccagaatcaacggatgaagtgtaagaaatggcaaaagaataactggccccgtaactccaatgggatgccccaaggtcccgctatggcagaatacccagggttctactcctaccaccagggctgcctggtaaacagccctggcaacctccctatgtggggcaaccagacttggaataaccctacatggtctaatcagagttggaactctcagagctggtctaatcactcttggaactcacaagcctggtgccctcaagcttggaataaccagccatggaataaccagttcaataactacatggaagagttcctgcagccaggtattcagctgcaacagaacagccccgtgtgtgacctggaggccaccctgggaaccgccggcgagaactataatgtgatccaacagaccgtcaagtatttcaattctcagcaacagatcacggacctgttcccgaactaccctctgaacatccagcctgaggacttgtaa

SEQ ID NO: 13 - protein, 506 aa, Bos taurus
MADAEVLILPKKHKKKKERKSLPEEAVAEIQHAEEFLIKPESRVAQLDTSQWPLLLKNFDKLNVRTTHYTPLPCGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWMQEYVDYSNSAKKDVPPKAVKATPVVAEVVKTPKRKRESESEDTSPAAPQVVKKEKKKKKKEKAKAAAEKPGAGDSDSTKKKKKKKIKAEMVSE

SEQ ID NO: 14 - DNA, 1521 bp, Bos taurus
  source 1..1521: mol_type = genomic DNA
atggcagatgctgaagttcttattctccctaagaaacacaaaaagaaaaaagaacgcaaatcactcccagaggaggccgttgcagagatacaacatgcggaagagttcctgatcaaaccagaatctagagttgctcaactggatacatctcaatggcctttgctgctgaaaaatttcgataaactcaacgtcagaacaacccactataccccgctcccgtgtgggtctaatccgcttaaacgagaaatcggtgactacattcggacaggttttattaatctcgataagcctagcaacccctctagtcacgaggttgtcgcttggatccgaaggatattgagggtcgagaagacagggcactcaggtacccttgatcccaaagtcaccggctgcctcatagtttgcatcgaaagagccactcgactggttaaatcccaacaatctgcaggaaaagagtatgttggcatagtcaggcttcataatgcgattgagggtggcacccaactctccagggcactcgaaacacttacgggggcacttttccagcgaccacctcttatagcggcagtcaaacggcaactccgagttaggacgatatacgagagcaagatgatcgagtatgatcctgagagacggcttggtatcttttgggttagttgtgaggccggcacgtatatccgaacgctttgtgtgcacttggggttgctgcttggggtcgggggccaaatgcaggaattgcggcgggtgcggtcaggagtgatgtcagagaaggaccatatggtgacgatgcatgacgtcctcgatgcccagtggctttacgacaatcacaaagatgagtcataccttaggcgagttgtctacccactcgaaaagttgctcacttcccacaaacgacttgtgatgaaagatagcgccgtgaacgcgatctgctatggcgctaagataatgctccccggtgtcctcagatacgaagacgggattgaagttaatcaagaaatcgttgtcattacgactaaaggagaggccatttgcatggcgatcgccctgatgacgactgccgtcataagtacatgcgatcatggaatagtggcaaaaatcaagagggtcatcatggaaagggacacatacccccggaaatggggtctgggaccgaaagcctctcaaaagaaactgatgattaaacagggacttcttgacaaacatggtaaaccgactgatagcactcccgctacatggatgcaagaatatgtggactattctaattccgctaaaaaggatgtgccgcctaaagcagtgaaagcgacccccgtcgttgctgaagtggttaaaacgccaaaacgcaaaagagagtctgaatcagaagacacttctcccgcagcgccgcaggtcgtcaaaaaggagaagaaaaaaaagaagaaagagaaggcgaaagctgccgctgagaagccgggtgccggtgacagcgatagtacaaagaagaaaaaaaaaaagaaaatcaaggctgaaatggtttctgagtag

SEQ ID NO: 15 - protein, 507 aa, Bos taurus
MDPGPPPPQPAPQGQGPPPAQAPQGQGPPSAPGQPAPPGPPAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAAAPAAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVLGGGSSNQQQQMRLQQLQMEKERLRLKQQELLRQVRPQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 16 - DNA, 1524 bp, Bos taurus
  source 1..1524: mol_type = genomic DNA
atggatcctggaccgcctcccccgcagcctgcgccccagggacaggggccacccccagcgcaggccccacaaggccagggtcccccaagcgctccaggccagcctgcaccacccggccctcccgctgcaccccaggccccacctgccggtcaccagatcgtccacgtccgcggggactccgagacagatctggaagctctctttaacgcggtgatgaacccaaagacggcaaatgtgcctcagaccgtccctatgcgcctgaggaagctgcccgactcctttttcaaacctccggagccaaaatcccattctcgccaggcctccacggatgccggcaccgcgggcgcactgacgccccagcacgtcagggctcattctagccctgccagccttcagctcggcgctgtgagccccgggaccctgacccctacgggagttgtgtccggccccgccgcagcccctgccgcacagcacctgcgccaatcctcttttgagattcccgatgacgtgcctctgcccgccggttgggagatggctaagacctctagcggccagaggtacttcctcaatcacattgaccagactaccacttggcaggacccccggaaggcgatgctttctcagatgaacgtgacagcgccgacttcccctcccgtccaacagaacatgatgaactccgccagcggcccactccccgatgggtgggagcaggctatgacccaggacggtgagatttattacatcaaccacaagaacaagacgacctcttggctggatccaagattggatccgcgcttcgccatgaaccagagaatctcacagtccgcccctgtgaagcagccgcccccgttggcgccgcaaagccctcagggcggagtgctgggcggtggatcttccaaccagcaacagcaaatgcgcttgcagcaattgcagatggaaaaagagaggctgcgcctcaaacaacaggaacttctgaggcaggtgcgcccccaggccatgaggaacatcaacccgtccaccgctaattctcctaagtgccaggaactggctctgcgctctcagctgcccacgctggagcaggacggcgggacccagaatcctgttagttctccgggaatgagccaagaactgcgtactatgactacaaacagctccgacccattcttgaacagcggcacctaccactctcgtgatgagagcaccgactcaggattgtccatgagctcctatagcgtgcctcgtacgcccgatgacttcctcaacagcgtggacgaaatggataccggtgacaccatcaaccaaagcaccctcccaagccagcaaaaccgtttcccagactacttggaggcaatcccggggacaaacgtggatcttggaactctggagggcgacgggatgaacatcgagggcgaagagctcatgccttccctccaggaggcgctgagctccgacatcctgaacgatatggaatccgtgttggctgcaaccaagctggacaaggagtccttcttgacatggttgtga

SEQ ID NO: 17 - DNA, 313 bp, synthetic construct
  source 1..313: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 18 - DNA, 165 bp, synthetic construct
  source 1..165: mol_type = other DNA
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaa